(19) United States
(12) Patent Application Publication
(10) Pub. No.: US 2010/0178679 A1
(43) Pub. Date: Jul. 15, 2010

(54) PRODUCTION OF ISOPRENOIDS

(75) Inventors: Larry Anthony, Aston, PA (US); Jack Newman, Berkeley, CA (US)

Correspondence Address: SHERIDAN ROSS PC, 1560 BROADWAY, SUITE 1200, DENVER, CO, 80202, US

(73) Assignee: Amyris Biotechnologies, Inc.

(21) Appl. No.: 12/595,600

(22) PCT Filed: Apr. 14, 2008
(86) PCT No.: PCT/US08/60199

§ 371 Date: Mar. 26, 2010

Related U.S. Application Data
(60) Provisional application No. 60/912,121, filed on Apr. 16, 2007.

(51) Int. Cl. C12P 5/02; C12N 1/15; C12N 1/21

(52) U.S. Cl. 435/167; 435/254.11; 435/252.33

(57) ABSTRACT

The present invention provides methods for a robust production of isoprenoids utilizing the DXP biosynthetic path-way. The invention also provides nucleic acids, enzymes, expression vectors, and genetically modified host cells for carrying out the subject methods.

### BACKGROUND OF THE INVENTION

Isoprenoids are ubiquitous in nature. They comprise a diverse family of over 40,000 individual products, many of which are vital to living organisms. Isoprenoids serve to maintain cellular fluidity, electron transport, and other metabolic functions. A vast number of natural and synthetic isoprenoids are useful as pharmaceuticals, cosmetics, perfumes, pigments and colorants, fungicides, antiseptics, nutraceuticals, and fine chemical intermediates.

An isoprenoid product is typically composed of repeating five carbon isopentenyl diphosphate (IPP) units, although irregular isoprenoids and polyterpenes have been reported. In nature, isoprenoids are synthesized by consecutive condensations of their precursor IPP and its isomer dimethylallyl pyrophosphate (DMAPP). Two pathways for these precursors are known. Eukaryotes, with the exception of plants, generally use the mevalonate-dependent (MEV) pathway to convert acetyl coenzyme A (acetyl-CoA) to IPP, which is subsequently isomerized to DMAPP. Prokaryotes, with some exceptions, typically employ only the mevalonate-independent or deoxyxylulose-5-phosphate (DXP) pathway to produce IPP and DMAPP. Plants use both the MEV pathway and the DXP pathway. See Rohmer et al. (1993) Biochem. J. 295:517-524; Lange et al. (2000) Proc. Natl. Acad. Sci. USA 97(24):13172-13177; Rohdich et al. (2002) Proc. Natl. Acad. Sci. USA 99:1158-1163.

Traditionally, isoprenoids have been manufactured by extraction from natural sources such as plants, microbes, and animals. However, the yield by way of extraction is usually very low due to a number of profound limitations. First, most isoprenoids accumulate in nature in only small amounts. Second, the source organisms in general are not amenable to the large-scale cultivation that is necessary to produce commercially viable quantities of a desired isoprenoid.

The elucidation of the MEV and DXP metabolic pathways has made biosynthetic production of isoprenoids feasible. For instance, microbes have been engineered to overexpress a part of or the entire MEV pathway for production of an isoprenoid named amorpha-4,11-diene (U.S. Pub. Nos. 20030148479 and 20060079476 by Keasling et al.).

In contrast to the MEV pathway, the genes encoding the DXP pathway have been only recently isolated (Rohmer, M. (1999), Nat. Prod. Rep. 16, 565, Lichtenthaler et al. (1997), J. Physiol. Plant. 101, 643-652. Eisenreich et al. (1998) Chem. Biol. 5, R221, Lange et al. (2000), Proc Natl Acad Sci USA. 97(24):13172). Still, some aspects of the DXP pathway still remains unknown including the mechanism by which 2C-methyl-D-erythritol-2,4-cyclodiphosphate is converted to 1-hydroxy-2-methyl-2-(E)-butenyl-4-diphosphate by the enzyme IspG. Despite incomplete knowledge of the DXP pathway, several publications have utilized the DXP pathway in E. coli to increase the intracellular concentration of FPP and thereby increase downstream production of isoprenoids, such as the C-40 carotenoids lycopene and β-carotene.

Some have focused on balancing the pool of glyceraldehyde-3-phosphate and pyruvate, or on increasing the expression of 1-deoxy-D-xylulose-5-phosphate synthase (dxs) and IPP isomerase (idi). See Farmer et al. (2001) Biotechnol. Prog. 17:57-61; Kajiwara et al. (1997) Biochem. J. 324:421-426; and Kim et al. (2001) Biotechnol. Bioeng. 72:408-415. Others have focused on the dxs and idi gene products which have been implicated as the rate-limiting enzymes in the DXP pathway (Harker and Bramley (1999) FEBS Lett. 448(1):115; Wang et al. (1999) Biotechnol. Bioeng. 62(2):235). To overcome this limitation, several studies have overexpressed these two genes in E. coli and have observed modest increases in isoprenoid production, primarily via an increase in beta-carotene production. (Albrecht et al. (1999) Biotech, Lett, 21: 791-795). More recent publications have attempted to increase production by placing the strong bacteriophage T5 promoter in front of the individual genes: dxs, ispD, ispF, idi, or ispB (Yuan et al. (2006) Metab Eng. 8(1):79).

While these efforts have shown some improvements, given the very large quantities of isoprenoid products needed for many commercial applications, there remains a need for expression systems that produce even more isoprenoids than available with current technologies. The present invention addresses this need and provides related advantages as well.

### SUMMARY OF THE INVENTION

The present invention relates to compositions and methods for the enhanced production of isoprenoid compounds by genetically engineered organisms containing a heterologous enzyme that converts IPP to DMAPP and heterologous enzymes for at least the first and seventh steps of DXP pathway. The DXP pathway offers potential advantages over the MEV pathway. First, the DXP pathway requires only one ATP per C5 isoprenoid unit versus three for the MEV pathway. In addition, starting materials for the DXP pathway, particularly pyruvate, are relatively more abundant in cells under both aerobic and anaerobic conditions than acetyl-CoA, the starting material for the MEV pathway. Consequently, the DXP pathway can be part of an effective strategy for cost-effective industrial scale isoprenoid production.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

FIG. 1 is a schematic representation of the 1-deoxy-D-xylulose 5-diphosphate (“DXP”) pathway for the production of isopentenyl pyrophosphate (“IPP”) and dimethylallyl pyrophosphate (“DMAPP”). Dxs is 1-deoxy-D-xylulose-5-phosphate synthase; Dxr is 1-deoxy-D-xylulose-5-phosphate reductoisomerase (also known as IspC); IspD is 4-diphosphocytidyl-2C-methyl-D-erythritol synthase; IspE is 4-diphosphocytidyl-2C-methyl-D-erythritol synthase; IspF is 2C-methyl-D-erythritol 2,4-cyclodiphosphate synthase; IspG is 1-hydroxy-2-methyl-2-(E)-butenyl 4-diphosphate synthase (IspG); and ispH is isopentenyl/dimethylallyl diphosphate synthase.

FIG. 2 is a schematic representation of the mevalonate (“MEV”) pathway for the production of isopentenyl pyrophosphate (“IPP”).

FIG. 3 is a schematic representation of the conversion of IPP and DMAPP to geranyl pyrophosphate (“GPP”), farnesyl pyrophosphate (“FPP”), and geranylgeranyl pyrophosphate (“GGPP”).

FIGS. 4A-V show certain nucleotide sequences used in the practice of the invention.

FIGS. 5A-C depict maps of expression plasmids pAM408, pAM409, and pAM424.

FIG. 6 depicts a map of expression plasmids pAM3 and pAM373.

FIG. 7 shows production of amorpha-4,11-diene by Escherichia coli host strains.

FIGS. 8A-B depict a nucleotide sequence encoding a β-farnesene synthase.

FIG. 9 shows production of β-farnesene synthase by Escherichia coli host strains.

### DETAILED DESCRIPTION OF THE INVENTION

### Definitions

Unless defined otherwise, all technical and scientific terms used herein have the same meaning as commonly understood by one of ordinary skill in the art to which this invention belongs. Reference is made here to a number of terms that shall be defined to have the following meanings:

The term “optional” or “optionally” means that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where the event or circumstance does not occur.

The term “metabolic pathway” is used herein to refer to a catabolic pathway or an anabolic pathway. Anabolic pathways involve constructing a larger molecule from smaller molecules, a process requiring energy. Catabolic pathways involve breaking down of larger molecules, often releasing energy.

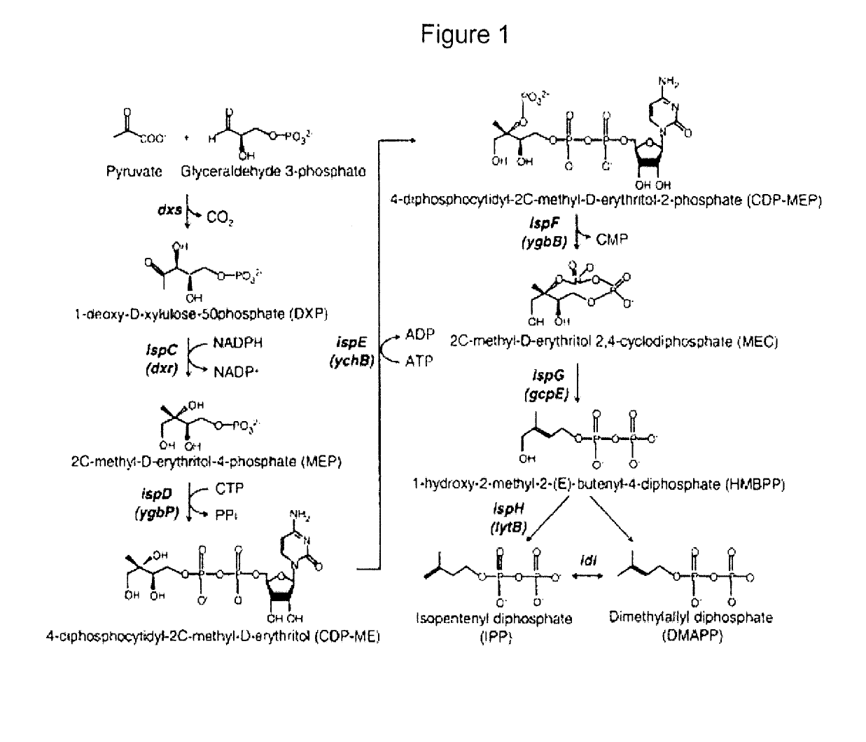

The term “deoxyxylulose 5-phosphate pathway” or “DXP pathway” is used herein to refer to the pathway that converts glyceraldehyde-3-phosphate and pyruvate to IPP and DMAPP. FIG. 1 illustrates the DXP pathway as well as the subsequent interconversion of IPP and DMAPP.

The word “pyrophosphate” is used interchangeably herein with “diphosphate”.

The terms “expression vector” or “vector” refer to a nucleic acid that transduces, transforms, or infects a host cell, thereby causing the cell to produce nucleic acids and/or proteins other than those that are native to the cell, or to express nucleic acids and/or proteins in a manner that is not native to the cell.

The term “endogenous” refers to a substance or process that occurs naturally, e.g., in a non-recombinant host cell.

The term “nucleic acid” refers to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically, or biochemically modified, non-natural, or derivatized nucleotide bases.

The term “operon” is used to refer to two or more contiguous nucleotide sequences that each encode a gene product such as a RNA or a protein, and the expression of which are coordinately regulated by one or more controlling elements (for example, a promoter).

The term “heterologous nucleic acid” as used herein refers to a nucleic acid wherein at least one of the following is true: (a) the nucleic acid is foreign (“exogenous”) to (that is, not naturally found in) a given host cell; (b) the nucleic acid comprises a nucleotide sequence that is naturally found in (that is, is “endogenous to”) a given host cell, but the nucleotide sequence is produced in an unnatural (for example, greater than expected or greater than naturally found) amount in the cell; (c) the nucleic acid comprises a nucleotide sequence that differs in sequence from an endogenous nucleotide sequence, but the nucleotide sequence encodes the same protein (having the same or substantially the same amino acid sequence) and is produced in an unnatural (for example, greater than expected or greater than naturally found) amount in the cell; or (d) the nucleic acid comprises two or more nucleotide sequences that are not found in the same relationship to each other in nature (for example, two or more gene sequences are placed closer together and/or in a different order than naturally found in the host cell).

The term “recombinant host” (also referred to as a “genetically modified host cell” or “genetically modified host microorganism”) denotes a host cell that comprises a heterologous nucleic acid of the invention.

The term “exogenous nucleic acid” refers to a nucleic acid that is exogenously introduced into a host cell, and hence is not normally or naturally found in and/or produced by a given cell in nature.

The term “regulatory element” refers to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” refers to a permanent or transient genetic change induced in a cell following introduction of new nucleic acid. Genetic change (“modification”) can be accomplished either by incorporation of the new DNA into the genome of the host cell, or by transient or stable maintenance of the new DNA as an episomal element. In eukaryotic cells, a permanent genetic change is generally achieved by introduction of the DNA into the genome of the cell. In prokaryotic cells, a permanent genetic change can be introduced into the chromosome or via extrachromosomal elements such as plasmids and expression vectors, which may contain one or more selectable markers to aid in their maintenance in the recombinant host cell.

The term “operably linked” refers to a juxtaposition wherein the components so described are in a relationship permitting them to function in their intended manner. For instance, a promoter is operably linked to a nucleotide sequence if the promoter affects the transcription or expression of the nucleotide sequence.

The term “host cell” and “host microorganism” are used interchangeably herein to refer to any archae, bacterial, or eukaryotic living cell into which a heterologous nucleic acid can be or has been inserted. The term also relates to the progeny of the original cell, which may not necessarily be completely identical in morphology or in genomic or total DNA complement to the original parent, due to natural, accidental, or deliberate mutation.

The term “naturally occurring” as applied to a nucleic acid, an enzyme, a cell, or an organism, refers to a nucleic acid, enzyme, cell, or organism that is found in nature. For example, a polypeptide or polynucleotide sequence that is present in an organism that can be isolated from a source in nature and that has not been intentionally modified by a human in the laboratory is naturally occurring.

The terms “isoprenoid”, “isoprenoid compound”, “isoprenoid product”, “terpene”, “terpene compound”, “terpenoid”, and “terpenoid compound” are used interchangeably herein. They refer to compounds that are capable of being derived from IPP. Exemplary isoprenoids include but are not limited to monoterpenes, diterpenes, sesquiterpenes, triterpenes, and polyterpenes.

The singular forms “a,” “and,” and “the” include plural referents unless the context clearly dictates otherwise. Thus, for example, reference to “an expression vector” includes a single expression vector as well as a plurality of expression vectors, and reference to “the host cell” includes reference to one or more host cells, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Unless otherwise indicated, this invention is not limited to particular sequences, expression vectors, enzymes, host microorganisms, or processes, as such may vary in accordance with the understanding of those of ordinary skill in the arts to which this invention pertains in view of the teaching herein. Terminology used herein is for purposes of describing particular embodiments only and is not intended to be limiting.

### DXP Pathway

The present invention relates to compositions and methods for the enhanced production of isoprenoid compounds by genetically engineered organisms containing a heterologous enzyme that converts IPP to DMAPP and heterologous enzymes for at least the first and seventh steps of DXP pathway. A schematic representation of the DXP pathway is shown in FIG. 1.

The DXP pathway combines pyruvate and D-glyceraldehyde 3-phosphate to make isopentyl pyrophosphate (isopentyl diphosphate) (IPP) and/or dimethylallyl pyrophosphate (dimethylallyl diphosphate) (DMAPP). The IPP and DMAPP are referred to as universal isoprenoid intermediates because they are intermediates to both the DXP and the MEV isoprenoid biosynthetic pathways. The DXP pathway comprises seven steps

In the first step, pyruvate is condensed with D-glyceraldehyde 3-phosphate to make 1-deoxy-D-xylulose-5-phosphate. An enzyme known to catalyze this step is, for example, 1-deoxy-D-xylulose-5-phosphate synthase. The gene encoding this enzyme is referred to as dxs. Illustrative examples of nucleotide sequences for dxs include but are not limited to: (AF035440; Escherichia coli), (NC_{—}002947, locus tag PPO527; Pseudomonas putida KT2440), (CP000026, locus tag SPA2301; Salmonella enterica subsp. enterica serovar Paratyphi A str. ATCC 9150), (NC_{—}007493, locus tag RSP_{—}0254; Rhodobacter sphaeroides 2.4.1), (NC_{—}005296, locus tag RPA0952; Rhodopseudomonas palustris CGA009), (NC_{—}004556, locus tag PD1293; Xylella fastidiosa Temeculal), (NC_{—}003076, locus tag AT5G11380; Arabidopsis thaliana), (Y18874, Synechococcus PCC6301), (AB026631, Streptomyces sp. CL190), (AB042821, Streptomyces griseolosporeus), (AF111814, Plasmodium falciparum), (AF143812, Lycopersicon esculentum), (AJ279019, Narcissus pseudonarcissus), and (AJ291721, Nicotiana tabacum).

In the second step, 1-deoxy-D-xylulose-5-phosphate is converted to 2C-methyl-D-erythritol-4-phosphate (MEP). An enzyme known to catalyze this step is, for example, 1-deoxy-D-xylulose-5-phosphate reductoisomerase. The gene encoding this enzyme is referred to as dxr or ispC. Illustrative examples of dxr or ispC nucleotide sequences include but are not limited to: (AB013300; Escherichia coli), (AF148852; Arabidopsis thaliana), (NC_{—}002947, locus tag PP1597; Pseudomonas putida KT2440), (AL939124, locus tag SCO5694; Streptomyces coelicolor A3(2)), (NC_{—}007493, locus tag RSP_{—}2709; Rhodobacter sphaeroides 2.4.1), (NC_{—}007492, locus tag Pfl_{—}1107; Pseudomonas fluorescens PfO-1), (AB049187, Streptomyces griseolosporeus), (AF111813, Plasmodium falciparum), (AF116825, Mentha×piperita), (AF148852, Arabidopsis thaliana), (AF182287, Artemisia annua), (AF250235, Catharanthus roseus), (AF282879, Pseudomonas aeruginosa) (AJ242588, Arabidopsis thaliana), (AJ250714, Zymomonas mobilis strain ZM4), (AJ292312, Klebsiella pneumoniae), (AJ297566, Zea mays).

In the third step, 2C-methyl-D-erythritol-4-phosphate (MEP) is converted to 4-diphosphocytidyl-2C-methyl-D-erythritol (CDP-ME). An enzyme known to catalyze this step is, for example, 4-diphosphocytidyl-2C-methyl-D-erythritol synthase. The gene encoding this enzyme is referred to as ispD or ygbP. Illustrative examples of ispD or ygbP nucleotide sequences include but are not limited to: (AF230736; Escherichia coli), (NC_{—}007493, locus tag RSP_{—}2835; Rhodobacter sphaeroides 2.4.1), (NC_{—}003071, locus tag AT2G02500; Arabidopsis thaliana), (AB037876, Arabidopsis thaliana), (AF109075, Clostridium difficile), (AF230736, Escherichia coli), and (AF230737, Arabidopsis thaliana).

In the fourth step, 4-diphosphocytidyl-2C-methyl-D-erythritol (CDP-ME) is converted to 4-diphosphocytidyl-2C-methyl-D-erythritol-2-phosphate (CDP-MEP). An enzyme known to catalyze this step is, for example, 4-diphosphocytidyl-2C-methyl-D-erythritol kinase. The gene encoding this enzyme is referred to as ispE or ychB. Illustrative examples of ispE or ychB nucleotide sequences include but are not limited to: (AF216300; Escherichia coli), (NC_{—}007493, locus_tag RSP_{—}1779; Rhodobacter sphaeroides 2.4.1), (AF263101, Lycopersicon esculentum), and (AF288615, Arabidopsis thaliana).

In the fifth step, 4-diphosphocytidyl-2C-methyl-D-erythritol-2-phosphate (CDP-MEP) is converted to 2C-methyl-D-erythritol 2,4-cyclodiphosphate (MEC). An enzyme known to catalyze this step is, for example, 2C-methyl-D-erythritol 2,4-cyclodiphosphate synthase. The gene encoding this enzyme is referred to as IspF or ygbB. Illustrative examples of IspF or ygbB nucleotide sequences include but are not limited to: (AF230738; Escherichia coli), (NC_{—}007493, locus_tag RSP_{—}6071; Rhodobacter sphaeroides 2.4.1), (NC_{—}002947, locus_tag PP1618; Pseudomonas putida KT2440), (AB038256, Escherichia coli mecs gene), (AF250236, Catharanthus roseus (MECS), (AF279661, Plasmodium falciparum), and (AF321531, Arabidopsis thaliana)

In the sixth step, 2C-methyl-D-erythritol 2,4-cyclodiphosphate (MEC) is converted to 1-hydroxy-2-methyl-2-(E)-butenyl-4-diphosphate (HMBPP). An enzyme known to catalyze this step is, for example, 1-hydroxy-2-methyl-2-(E)-butenyl-4-diphosphate synthase. The gene encoding this enzyme is referred to as ispG or gcpE. Illustrative examples of ispG or gcpE nucleotide sequences include but are not limited to: (AY033515; Escherichia coli), (NC_{—}002947, locus_tag PP0853; Pseudomonas putida KT2440), (NC_{—}007493, locus_tag RSP_{—}2982; Rhodobacter sphaeroides 2.4.1), (067496, Aquifex aeolicus), (P54482, Bacillus subtilis), (Q9pky3, Chlamydia muridarum), (Q9Z8H0, Chlamydophila pneumoniae), (084060, Chlamydia trachomatis), (P27433, Escherichia coli), (P44667, Haemophilus influenzae), (Q9ZLL0, Helicobacter pylori J99), (O33350, Mycobacterium tuberculosis), (S77159, Synechocystis sp.), (Q9WZZ3, Thermotoga maritima), (O83460, Treponema pallidum), (Q9JZ40, Neisseria meningitidis), (Q9PPM1, Campylobacter jejuni), (Q9RXC9, Deinococcus radiodurans), (AAG07190, Pseudomonas aeruginosa) and (Q9KTX1, Vibrio cholerae).

In the seventh step, 1-hydroxy-2-methyl-2-(E)-butenyl-4-diphosphate (HMBPP) is converted into either isopentyl pyrophosphate (IPP) or its isomer, dimethylallyl diphosphate (DMAPP). An enzyme known to catalyze this step is, for example, isopentyl/dimethylallyl diphosphate synthase. The gene encoding this enzyme is referred to as ispH or 1ytB. Illustrative examples of nucleotide sequences include but are not limited to: (AY062212; Escherichia coli), (NC_{—}002947, locus_tag PPO606; Pseudomonas putida KT2440), (AF027189, Acinetobacter sp. BD413), (AF098521, Burkholderia pseudomallei), (AF291696, Streptococcus pneumoniae), (AF323927, Plasmodium falciparum gene), (M87645, Bacillus subtillis), (U38915, Synechocystis sp.), and (X89371, C. jejunisp O67496)

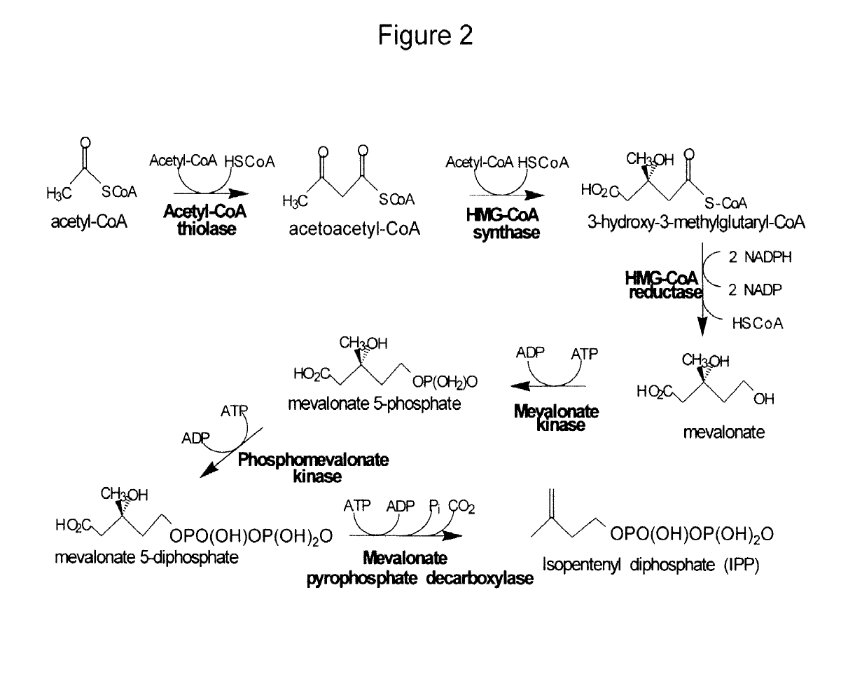

The IPP and DMAPP are interconverted enzymatically. An enzyme known to catalyze this step is, for example, IPP isomerase. The gene encoding this enzyme is referred to as idi. Illustrative examples of idi nucleotide sequences include but are not limited to: (NC_{—}000913, 3031087.3031635; Escherichia coli), and (AF082326; Haematococcus pluvialis). The heterologous expression of IPP isomerase in the present invention ensures that the conversion of IPP into DMAPP does not represent a rate-limiting step in the overall pathway. IPP and DMAPP can be converted to isoprenoid compounds containing more than five carbons via condensation. FIG. 2 illustrates various condensation products such as geranyl pyrophosphate, farnesyl pyrophosphate and geranylgeranyl pyrophosphate which are in turn modified to monoterpenes, sesquiterpenes, diterpenes and carotenoids.

### Engineering Pathways

The present invention relates to compositions and methods for the enhanced production of isoprenoid compounds by genetically engineered organisms containing a heterologous enzyme that converts IPP to DMAPP and heterologous enzymes for at least the first and seventh steps of DXP pathway.

In one aspect of the invention, a genetically modified host cell capable of converting pyruvate and glyceraldehyde 3-phosphate into dimethylallydiphosphate (DMAPP) is provided. The host cell comprises heterologous nucleic acid sequences that encode:
a. an enzyme that converts pyruvate and glyceraldehyde 3-phosphate to 1-deoxy-D-xylulose-5-phosphate (DXP);
b. an enzyme that converts 1-hydroxy-2-methyl-2-(E)-butenyl-4-diphosphate (HMBPP) to IPP, and,
c. an enzyme that converts IPP to DMAPP.

In some embodiments, the host cell additionally comprises one or more additional heterologous nucleic acid sequences that encode one or more enzymes selected from the group consisting of:
a. an enzyme that converts DXP to 2C-methyl-D-erythritol-4-phosphate (MEP);
b. an enzyme that converts MEP to 4-diphosphocytidyl-2C-methyl-D-erythritol (CDP-ME);
c. an enzyme that converts CDP-ME to 4-diphosphocytidyl-2C-methyl-D-erythritol-2-phosphate (CDP-MEP);
d. an enzyme that converts CDP-MEP to 2C-methyl-D-erythritol 2,4-cyclodiphosphate (MEC); and
e. an enzyme that converts MEC to HMBPP.

In other embodiments, the host cell comprises one additional heterologous nucleic acid sequence that encodes an enzyme selected from the group consisting of an enzyme that converts DXP to MEP; an enzyme that converts MEP to CDP-ME; an enzyme that converts CDP-ME to CDP-MEP; and enzyme that converts CDP-MEP to MEC; and an enzyme that converts MEC to HMBPP.

In other embodiments, the host cell comprises two additional heterologous nucleic acid sequences that encode enzymes selected from the group consisting of: an enzyme that converts DXP to MEP; an enzyme that converts MEP to CDP-ME; an enzyme that converts CDP-ME to CDP-MEP; and enzyme that converts CDP-MEP to MEC; and an enzyme that converts MEC to HMBPP

In other embodiments, the host cell comprises three additional heterologous nucleic acid sequences that encode enzymes selected from the group consisting of: an enzyme that converts DXP to MEP; an enzyme that converts MEP to CDP-ME; an enzyme that converts CDP-ME to CDP-MEP; and enzyme that converts CDP-MEP to MEC; and an enzyme that converts MEC to HMBPP

In other embodiments, the host cell comprises additional heterologous nucleic acid sequences that encode: an enzyme that converts DXP to MEP; an enzyme that converts MEP to CDP-ME; an enzyme that converts CDP-ME to CDP-MEP; and enzyme that converts CDP-MEP to MEC; and an enzyme that converts MEC to HMBPP

In some embodiments, the host cells comprise an endogenous DXP pathway in addition to the heterologous nucleic acid sequences that encode heterologous DXP pathway enzymes. In other embodiments, the endogenous DXP pathway has been functionally disabled. In other embodiments, the host cells comprise an endogenous MEV pathway (as illustrated by FIG. 2). In still other embodiments, the host cells comprise an endogenous MEV pathway that has been functionally disabled. The DXP or MEV endogenous pathway can be functionally disabled by disabling gene expression or inactivating the function of one or more of the pathway enzymes.

In another aspect, a genetically modified host cell having an endogenous DXP pathway is provided. The host cell comprises heterologous nucleic acid sequences wherein the heterologous nucleic acid sequences encode:
a. an enzyme that converts pyruvate and glyceraldehyde 3-phosphate to 1-deoxy-D-xylulose-5-phosphate (DXP);
b. an enzyme that converts DXP to 2C-methyl-D-erythritol-4-phosphate (MEP);
c. an enzyme that converts MEP to 4-diphosphocytidyl-2C-methyl-D-erythritol (CDP-ME);
d. an enzyme that converts CDP-ME to 4-diphosphocytidyl-2C-methyl-D-erythritol-2-phosphate (CDP-MEP);
e. an enzyme that converts CDP-MEP to 2C-methyl-D-erythritol 2,4-cyclodiphosphate (MEC);
f. an enzyme that converts MEC to 1-hydroxy-2-methyl-2-(E)-butenyl-4-diphosphate (HMBPP);
g. an enzyme that converts HMBPP to IPP, and,
h. an enzyme that converts IPP to DMAPP.

Other aspects of the invention include vectors comprising the heterologous nucleic acid sequences as well as methods for using the above genetically modified host cells to make isoprenoid or isoprenoid precursors,

The heterologous nucleic acid sequences of the present invention can be expressed by a single or multiple vectors. The nucleic acid sequences can be arranged in any order in a single operon, or in separate operons that are placed in one or multiple vectors. Where desired, two or more expression vectors can be employed, each of which contains one or more heterologous sequences operably linked in a single operon. While the choice of single or multiple vectors and the use of single or multiple promoters may depend on the size of the heterologous sequences and the capacity of the vectors, it will largely dependent on the overall yield of a given isoprenoid that the vector is able to provide when expressed in a selected host cell. In some instances, two-operon expression system provides a higher yield of isoprenoid. The subject vectors can stay replicable episomally, or as an integral part of the host cell genome.

In certain embodiments, the heterologous nucleic acids of the present invention are under the control of a single regulatory element. In some cases, the heterologous nucleic acid sequences are regulated by a single promoter. In other cases, the heterologous nucleic acid sequences are placed within a single operon. In still other cases, the heterologous nucleic acid sequences are placed within a single reading frame.

Where desired, the subject nucleic acid sequences can be modified to reflect the codon preference of a selected host cell to effect a higher expression of such sequences in a host cell. For example, the subject nucleotide sequences will in some embodiments be modified for yeast codon preference. See, e.g., Bennetzen and Hall (1982) J: Biol. Chem. 257(6): 3026-3031. As another non-limiting example, the nucleotide sequences will in other embodiments be modified for E. coli codon preference. See, e.g., Gouy and Gautier (1982) Nucleic Acids Res. 10(22):7055-7074; Eyre-Walker (1996) Mol. Biol. Evol. 13(6):864-872. See also Nakamura et al. (2000) Nucleic Acids Res. 28(1):292. Codon usage tables for many organisms are available, which can be used as a reference in designing sequences of the present invention. The use of prevalent codons of a given host microorganism generally increases the likelihood of translation, and hence the expression level of the desired sequences. Preparation of the subject nucleic acids can be carried out by a variety of routine recombinant techniques and synthetic procedures. Standard recombinant DNA and molecular cloning techniques used in the Examples are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987). Briefly, the subject nucleic acids can be prepared genomic DNA fragments, cDNAs, and RNAs, all of which can be extracted directly from a cell or recombinantly produced by various amplification processes including but not limited to PCR and rt-PCR.

Direct chemical synthesis of nucleic acids typically involves sequential addition of 3′-blocked and 5′-blocked nucleotide monomers to the terminal 5′-hydroxyl group of a growing nucleotide polymer chain, wherein each addition is effected by nucleophilic attack of the terminal 5′-hydroxyl group of the growing chain on the 3′-position of the added monomer, which is typically a phosphorus derivative, such as a phosphotriester, phosphoramidite, or the like. Such methodology is known to those of ordinary skill in the art and is described in the pertinent texts and literature (for example, Matteuci et al. (1980) Tet. Lett. 521:719; U.S. Pat. No. 4,500,707 to Caruthers et al.; and U.S. Pat. Nos. 5,436,327 and 5,700,637 to Southern et al.).

The invention relates in some embodiments to an increase in the level of transcription of nucleic acids in a host organism. The level of transcription of a nucleic acid in a host microorganism can be increased in a number of ways. For example, this can be achieved by increasing the copy number of the nucleotide sequence encoding the enzyme (e.g., by using a higher copy number expression vector comprising a nucleotide sequence encoding the enzyme, or by introducing additional copies of a nucleotide sequence encoding the enzyme into the genome of the host microorganism, for example, by recA-mediated recombination, use of “suicide” vectors, recombination using lambda phage recombinase, and/or insertion via a transposon or transposable element). In addition, it can be carried out by changing the order of the coding regions on the polycistronic mRNA of an operon or breaking up an operon into individual genes, each with its own control elements, or increasing the strength of the promoter (transcription initiation or transcription control sequence) to which the enzyme coding region is operably linked (for example, using a consensus arabinose- or lactose-inducible promoter in an Escherichia coli host microorganism in place of a modified lactose-inducible promoter, such as the one found in pBluescript and the pBBR1MCS plasmids), or using an inducible promoter and inducing the inducible-promoter by adding a chemical to a growth medium.

The level of translation of a nucleotide sequence in a host microorganism can be increased in a number of ways, including, but not limited to, increasing the stability of the mRNA, modifying the sequence of the ribosome binding site, modifying the distance or sequence between the ribosome binding site and the start codon of the enzyme coding sequence, modifying the entire intercistronic region located “upstream of” or adjacent to the 5′ side of the start codon of the enzyme coding region, stabilizing the 3′-end of the mRNA transcript using hairpins and specialized sequences, modifying the codon usage of enzyme, altering expression of rare codon tRNAs used in the biosynthesis of the enzyme, and/or increasing the stability of the enzyme, as, for example, via mutation of its coding sequence. Determination of preferred codons and rare codon tRNAs can be based on a sequence analysis of genes derived from the host microorganism.

The activity of a DXP pathway enzyme or prenyltransferase in a host can be altered in a number of ways, including, but not limited to, expressing a modified form of the enzyme that exhibits increased solubility in the host cell, expressing an altered form of the enzyme that lacks a domain through which the activity of the enzyme is inhibited, expressing a modified form of the enzyme that has a higher Kcat or a lower Km for the substrate, or expressing an altered form of the enzyme that is not affected by feed-back or feed-forward regulation by another molecule in the pathway. Such variant enzymes can also be isolated through random mutagenesis of a broader specificity enzyme, and a nucleotide sequence encoding such variant enzyme can be expressed from an expression vector or from a recombinant gene integrated into the genome of a host microorganism.

The subject vector can be constructed to yield a desired level of copy numbers of the encoded enzyme. In some embodiments, the subject vectors yield at least 10, between 10 to 20, between 20-50, between 50-100, or even higher than 100 copies of the desired enzymes. Low copy number plasmids generally provide fewer than about 20 plasmid copies per cell; medium copy number plasmids generally provide from about 20 plasmid copies per cell to about 50 plasmid copies per cell, or from about 20 plasmid copies per cell to about 80 plasmid copies per cell; and high copy number plasmids generally provide from about 80 plasmid copies per cell to about 200 plasmid copies per cell, or more.

Suitable low copy expression vectors for Escherichia coli include, but are not limited to, pACYC184, pBeloBac11, pBR332, pBAD33, pBBR1MCS and its derivatives, pSC101, SuperCos (cosmid), and pWE15 (cosmid). Suitable medium copy expression vectors for Escherichia coli include, but are not limited to pTrc99A, pBAD24, and vectors containing a ColE 1 origin of replication and its derivatives. Suitable high copy number expression vectors for Escherichia coli include, but are not limited to, pUC, pBluescript, pGEM, and pTZ vectors. Suitable low-copy (centromeric) expression vectors for yeast include, but are not limited to, pRS415 and pRS416 (Sikorski & Hieter (1989) Genetics 122:19-27). Suitable high-copy 2 micron expression vectors in yeast include, but are not limited to, pRS425 and pRS426 (Christainson et al. (1992) Gene 110:119-122). Alternative 2 micron expression vectors include non-selectable variants of the 2 micron vector (Bruschi & Ludwig (1988) Curr. Genet. 15:83-90) or intact 2 micron plasmids bearing an expression cassette (as exemplified in U.S. Pat. Appl. 20050084972) or 2 micron plasmids bearing a defective selection marker such as LEU2d (Erhanrt et al. (1983) J. Bacteriol. 156 (2): 625-635) or URA3d (Okkels (1996) Annals of the New York Academy of Sciences 782(1): 202-207).

Regulatory elements include, for example, promoters and operators, which can also be engineered to increase the metabolic flux of the DXP pathways by increasing the expression of one or more genes that play a significant role in determining the overall yield of an isoprenoid produced. A promoter is a sequence of nucleotides that initiates and controls the transcription of a nucleic acid sequence by an RNA polymerase enzyme. An operator is a sequence of nucleotides adjacent to the promoter that functions to control transcription of the desired nucleic acid sequence. The operator contains a protein-binding domain where a specific repressor protein can bind. In the absence of a suitable repressor protein, transcription initiates through the promoter. In the presence of a suitable repressor protein, the repressor protein binds to the operator and thereby inhibits transcription from the promoter.

In some embodiments of the present invention, promoters used in expression vectors are inducible. In other embodiments, the promoters used in expression vectors are constitutive. In some embodiments, one or more nucleic acid sequences are operably linked to an inducible promoter, and one or more other nucleic acid sequences are operably linked to a constitutive promoter.

Non-limiting examples of suitable promoters for use in prokaryotic host cells include a bacteriophage T7 RNA polymerase promoter; a trp promoter; a lac operon promoter; a hybrid promoter, for example, a lac/tac hybrid promoter, a tac/trc hybrid promoter, a trp/lac promoter, a T7/lac promoter; a trc promoter; a tac promoter, and the like; an araBAD promoter; in vivo regulated promoters, such as an ssaG promoter or a related promoter (see, for example, U.S. Patent Publication No. 20040131637), a pagC promoter (Pulkkinen and Miller, J. Bacteriol. (1991) 173(1):86-93; Alpuche-Aranda et al. (1992) Proc. Natl. Acad. Sci. USA. 89(21):10079-83), a nirB promoter (Harborne et al. (1992) Mol. Micro. 6:2805-2813), and the like (see, for example, Dunstan et al. (1999) Infect. Immun. 67:5133-5141; McKelvie et al. (2004) Vaccine 22:3243-3255; and Chatfield et al. (1992) Biotechnol. 10:888-892); a sigma70 promoter, for example, a consensus sigma70 promoter (see, for example, GenBank Accession Nos. AX798980, AX798961, and AX798183); a stationary phase promoter, for example, a dps promoter, an spy promoter, and the like; a promoter derived from the pathogenicity island SPI-2 (see, for example, WO96/17951); an actA promoter (see, for example, Shetron-Rama et al. (2002) Infect. Immun. 70:1087-1096); an rpsM promoter (see, for example, Valdivia and Falkow (1996) Mol. Microbiol. 22:367 378); a tet promoter (see, for example, Hillen et al. (1989) In Saenger W. and Heinemann U. (eds) Topics in Molecular and Structural Biology, Protein-Nucleic Acid Interaction. Macmillan, London, UK, Vol. 10, pp. 143-162); an SP6 promoter (see, for example, Melton et al. (1984) Nucl. Acids Res. 12:7035-7056); and the like.

In some embodiment, the total activity of a heterologous enzymes that plays a larger role in the overall yield of an isoprenoid relative to other enzymes in the respective pathways is increased by expressing the enzyme from a strong promoter. Suitable strong promoters for Escherichia coli include, but are not limited to Trc, Tac, T5, T7, and P_{Lambda}. In another embodiment of the present invention, the total activity of the one or more MEV pathway enzymes in a host is increased by expressing the enzyme from a strong promoter on a high copy number plasmid. Suitable examples, for Escherichia coli include, but are not limited to using Trc, Tac, T5, T7, and P_{Lambda} promoters with pBAD24, pBAD 18, pGEM, pBluescript, pUC, and pTZ vectors.

Non-limiting examples of suitable promoters for use in eukaryotic host cells include, but are not limited to, a CMV immediate early promoter, an HSV thymidine kinase promoter, an early or late SV40 promoter, LTRs from retroviruses, and a mouse metallothionein-I promoter.

Non-limiting examples of suitable constitutive promoters for use in prokaryotic host cells include a sigma70 promoter (for example, a consensus sigma70 promoter). Non-limiting examples of suitable inducible promoters for use in bacterial host cells include the pL of bacteriophage λ; Plac; Ptrp; Ptac (Ptrp-lac hybrid promoter); an isopropyl-beta-D44 thiogalactopyranoside (IPTG)-inducible promoter, for example, a lacZ promoter; a tetracycline inducible promoter; an arabinose inducible promoter, for example, PBAD (see, for example, Guzman et al. (1995) J. Bacteriol. 177:4121-4130); a xylose-inducible promoter, for example, Pxyl (see, for example, Kim et al. (1996) Gene 181:71-76); a GAL1 promoter; a tryptophan promoter; a lac promoter; an alcohol-inducible promoter, for example, a methanol-inducible promoter, an ethanol-inducible promoter; a raffinose-inducible promoter; a heat-inducible promoter, for example, heat inducible lambda PL promoter; a promoter controlled by a heat-sensitive repressor (for example, CI857-repressed lambda-based expression vectors; see, for example, Hoffmann et al. (1999) FEMS Microbiol Lett. 177(2):327-34); and the like.

Non-limiting examples of suitable constitutive promoters for use in yeast host cells include an ADH1, an ADH2, a PGK, or a LEU2 promoter. Non-limiting examples of suitable inducible promoters for use in yeast host cells include, but are not limited to, a divergent galactose-inducible promoter such as a GAL 1 or a GAL 10 promoter (West at al. (1984) Mol. Cell. Biol. 4(11):2467-2478), or a CUP1 promoter. Where desired, the subject vector comprise a promoter that is stronger than a native E. Coli Lac promoter.

Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, for example, deBoer et al. (1983) Proc. Natl. Acad. Sci. U.S.A. 80:21-25.).

The genes in the expression vector typically will also encode a ribosome binding site to direct translation (that is, synthesis) of any encoded mRNA gene product. For suitable ribosome binding sites for use in Escherichia coli, see Shine et al. (1975) Nature 254:34, and Steitz, in Biological Regulation and Development: Gene Expression (ed. R. F. Goldberger), vol. 1, p. 349, 1979, Plenum Publishing, N.Y. Insertion of the ribosome binding site encoding nucleotide sequence such as 5′-AAAACA-3′ upstream of a coding sequence facilitates efficient translation in a yeast host microorganism (Looman et al. (1993) Nuc. Ac. Res. 21:4268-4271; Yun et. al. (1996) Mol. Microbiol. 19:1225-1239). In some embodiments of the invention, optimized Shine-Dalgarno sequences are inserted upstream of the coding region of a gene that is placed within a DXP module. The optimized Shine-Dalgarno sequences ensure efficient ribosome binding and translation of the coding sequence.

Other regulatory elements that may be used in an expression vector include transcription enhancer elements and transcription terminators. See, for example, Bitter et al. (1987) Methods in Enzymology, 153:516-544.

An expression vector may be suitable for use in particular types of host microorganisms and not others. One of ordinary skill in the art, however, can readily determine through routine experimentation whether a particular expression vector is suited for a given host microorganism. For example, the expression vector can be introduced into the host organism, which is then monitored for viability and expression of any genes contained in the vector.

The expression vector may also contain one or more selectable marker genes that, upon expression, confer one or more phenotypic traits useful for selecting or otherwise identifying host cells that carry the expression vector. Non-limiting examples of suitable selectable markers for eukaryotic cells include dihydrofolate reductase and neomycin resistance. Non-limiting examples of suitable selectable markers for prokaryotic cells include tetracycline, ampicillin, chloramphenicol, carbenicillin, and kanamycin resistance.

For production of isoprenoid at an industrial scale, it may be impractical or too costly to use a selectable marker that requires the addition of an antibiotic to the fermentation media. Accordingly, some embodiments of the present invention employ host cells that do not require the use of an antibiotic resistance conferring selectable marker to ensure plasmid (expression vector) maintenance. In these embodiments of the present invention, the expression vector contains a plasmid maintenance system such as the 60-kb IncP (RK2) plasmid, optionally together with the RK2 plasmid replication and/or segregation system, to effect plasmid retention in the absence of antibiotic selection (see, for example, Sia et al. (1995) J. Bacteriol. 177:2789-97; Pansegrau et al. (1994) J. Mol. Biol. 239:623-63). A suitable plasmid maintenance system for this purpose is encoded by the parDE operon of RK2, which codes for a stable toxin and an unstable antitoxin. The antitoxin can inhibit the lethal action of the toxin by direct protein-protein interaction. Cells that lose the expression vector that harbors the parDE operon are quickly deprived of the unstable antitoxin, resulting in the stable toxin then causing cell death. The RK2 plasmid replication system is encoded by the trfA gene, which codes for a DNA replication protein. The RK2 plasmid segregation system is encoded by the parCBA operon, which codes for proteins that function to resolve plasmid multimers that may arise from DNA replication.

The subject vectors can be introduced into a host cell stably or transiently by variety of established techniques. For example, one method involves a calcium chloride treatment wherein the expression vector is introduced via a calcium precipitate. Other salts, for example calcium phosphate, may also be used following a similar procedure. In addition, electroporation (that is, the application of current to increase the permeability of cells to nucleic acids) may be used. Other transformation methods include microinjection, DEAE dextran mediated transformation, and heat shock in the presence of lithium acetate. Lipid complexes, liposomes, and dendrimers may also be employed to transfect the host microorganism.

Upon transformation, a variety of methods can be practiced to identify the host cells into which the subject vectors have been introduced. One exemplary selection method involves subculturing individual cells to form individual colonies, followed by testing for expression of the desired gene product. Another method entails selecting transformed host cells based upon phenotypic traits conferred through the expression of selectable marker genes contained within the expression vector. Those of ordinary skill can identify genetically modified host cells using these or other methods available in the art.

In some embodiments, the heterlogous nucleic acid sequences can be inserted into the genome of the host organism. One method useful for the introduction of such sequences is the use of integration cassettes. Integration cassettes are typically linear double-stranded DNA fragments which can be chromosomally integrated by homologous recombination via the use of two PCR-generated fragments or one PCR-generated fragment. The integration cassette comprises a nucleic acid integration fragment that contains an expressible DNA fragment and a selectable marker bounded by specific recombinase sites responsive to a site-specific recombinase, and homology arms having homology to different portions of the host cell's chromosome. (see, for example, US Patent Application 2004/0219629). Generally, the preferred length of the homology arms is about 10 to about 100 base pairs in length. From 20 to 40 base pairs of homology, the efficiency of homologous recombination increases by four orders of magnitude (Yu et al. PNAS. 97:5978-5983. (2000)). One method of introducing DXP modules into the host genome utilizes the λ-Red recombinase system. The λ-Red system enables the use of homologous recombination as a tool for in vivo chromosomal engineering in hosts, such as E. coli, normally considered difficult to transform by homologous recombination. The λ-Red system works in other bacteria as well (Poteete, A., supra, 2001). Use of the λ-Red recombinase system can be applicable to other hosts generally used for industrial production.

The introduction of various pathway sequences of the invention into a host cell can be confirmed by methods such as PCR, Southern blot or Northern blot hybridization. For example, nucleic acids can be prepared from the resultant host cells, and the specific sequences of interest can be amplified by PCR using primers specific for the sequences of interest. The amplified product is subjected to agarose gel electrophoresis, polyacrylamide gel electrophoresis or capillary electrophoresis, followed by staining with ethidium bromide, SYBR Green solution or the like, or detection of DNA with a UV detection. Alternatively, nucleic acid probes specific for the sequences of interest can be employed in a hybridization reaction. The expression of a specific gene sequence can be ascertained by detecting the corresponding mRNA via reveres-transcription coupled PCR, Northern blot hybridization, or by immunoassays using antibodies reactive with the encoded gene product. Exemplary immunoassays include but are not limited to ELISA, radioimmunoassays, and sandwich immunoassays.

The enzymatic activity of a given pathway enzyme can be assayed by a variety of methods known in the art. In general, the enzymatic activity can be ascertained by the formation of the product or conversion of a substrate of an enzymatic reaction that is under investigation. The reaction can take place in vitro or in vivo.

The yield of an isoprenoid via one or more metabolic pathways disclosed herein can be augmented by inhibiting reactions that divert intermediates from productive steps towards formation of the isoprenoid product. Inhibition of the unproductive reactions can be achieved by reducing the expression and/or activity of enzymes involved in one or more unproductive reactions. Such reactions include side reactions of the TCA cycle that lead to fatty acid biosynthesis, alanine biosynthesis, the aspartate superpathway, gluconeogenesis, heme biosynthesis, and/or glutamate biosynthesis, at a level that affects the overall yield of an isoprenoid production Inhibition can be accomplished by reducing or eliminating the expression of certain genes in the target pathway or in competing pathways that may serve as competing sinks for energy or carbon. Where the sequence of the gene to be disrupted is known, one effective method of gene down-regulation is targeted gene disruption, where foreign DNA is inserted into a structural gene so as to disrupt transcription. This can be effected by the creation of genetic cassettes comprising the DNA to be inserted (often a genetic marker) flanked by sequence having a high degree of homology to a portion of the gene to be disrupted. Introduction of the cassette into the host cell results in insertion of the foreign DNA into the structural gene via the native DNA replication mechanisms of the cell or by the λ-Red recombination system. (See for example Hamilton et al., J. Bacteriol., 171:4617-4622 (1989); Balbas et al., Gene, 136:211-213 (1993); Gueldener et al., Nucleic Acids Res., 24:2519-2524 (1996); and Smith et al., Methods Mol. Cell. Biol., 5:270-277 (1996)). Antisense technology is another method of down regulating genes where the sequence of the target gene is known. To accomplish this, a nucleic acid segment from the desired gene is cloned and operably linked to a promoter such that the anti-sense strand of RNA will be transcribed. This construct is then introduced into the host cell and the antisense strand of RNA is produced. Antisense RNA inhibits gene expression by preventing the accumulation of mRNA which encodes the protein of interest. A person of skill in the art will know that special considerations are associated with the use of antisense technologies in order to reduce expression of particular genes. For example, the proper level of expression of antisense genes may require the use of different chimeric genes utilizing different regulatory elements known to the skilled artisan.

Other less specific methodologies can also be used to down regulate undesired activity. For example, cells may be exposed to UV radiation and then screened for the desired phenotype. Mutagenesis with chemical agents can also be effective for generating mutants and commonly used substances include chemicals that affect non-replicating DNA such as HNO₂ and NH₂OH, as well as agents that affect replicating DNA such as acridine dyes, notable for causing frame-shift mutations. Specific methods for creating mutants using radiation or chemical agents are well documented in the art. See for example Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36, 227, (1992).

Another non-specific method of gene disruption is the use of transposable elements or transposons. Transposons are genetic elements that insert randomly into DNA but can be later retrieved on the basis of sequence to determine where the insertion has occurred. Both in vivo and in vitro transposition methods are known. Both methods involve the use of a transposable element in combination with a transposase enzyme. When the transposable element or transposon is contacted with a nucleic acid fragment in the presence of the transposase, the transposable element will randomly insert into the nucleic acid fragment. The technique is useful for random mutagenesis and for gene isolation, since the disrupted gene may be identified on the basis of the sequence of the transposable element. Kits for in vitro transposition are commercially available (see for example The Primer Island Transposition Kit, available from Perkin Elmer Applied Biosystems, Branchburg, N.J., based upon the yeast Ty1 element; The Genome Priming System, available from New England Biolabs, Beverly, Mass.; based upon the bacterial transposon Tn7; and the EZ::TN Transposon Insertion Systems, available from Epicentre Technologies, Madison, Wis., based upon the Tn5 bacterial transposable element). Transposon-mediated random insertion in the chromosome can be used for isolating mutants for any number of applications including enhanced production of any number of desired products including enzymes or other proteins, amino acids, or small organic molecules including alcohols.

An example where the reduction of a side reaction can increased the levels of a desired isoprenoid compound is that mediated by squalene synthase in those host cells with an endogenous mevalonate pathway such as yeast. In such systems, erg9 mutants that have a reduced ability to convert FPP into squalene have been shown to make more FPP-derived isoprenoid product (see e.g., Karst and Lacroute, Molec. Gen. Genet., 154, 269-277 (1977); U.S. Pat. No. 5,589,372). Where the erg9 gene is blocked in yeast, such erg9 mutants may need extraneous ergosterol or other sterols added to the medium for the cells to remain viable because yeast strains generally need ergosterol for cell membrane fluidity. The cells normally cannot utilize this additional sterol unless grown under anaerobic conditions. However, erg 9 mutants which takes up exogenously supplied sterols under aerobic conditions have been identified. These include those having genetic modifications in upc (uptake control mutation which allows cells to take up sterols under aerobic conditions); hem1 (the HEM1 gene encodes aminolevulinic acid synthase which is the first committed step to the heme biosynthetic pathway from FPP, and hem1 mutants are capable of taking up ergosterol under aerobic conditions following a disruption in the ergosterol biosynthetic pathway, provided the cultures are supplemented with unsaturated fatty acids); and overexpression of the SUT1 (sterol uptake) gene can be used to allow for uptake of sterols under aerobic conditions (Bourot and Karst, Gene, 165: 97-102 (1995)).

### Host Cells

A wide variety of host cell can be used in the practice of the present invention. In one embodiment, the host cell is a genetically modified host microorganism in which nucleic acid molecules have been inserted, deleted or modified (i.e., mutated; e.g., by insertion, deletion, substitution, and/or inversion of nucleotides), to produce the desired isoprenoid compound or isoprenoid derivative

Illustrative examples of suitable host cells include any archae, bacterial, or eukaryotic cell. Examples of an archae cell include, but are not limited to those belonging to the genera: Aeropyrum, Archaeglobus, Halobacterium, Methanococcus, Methanobacterium, Pyrococcus, Sulfolobus, and Thermoplasma. Illustrative examples of archae strains include but are not limited to: Aeropyrum pernix, Archaeoglobus fulgidus, Methanococcus jannaschii, Methanobacterium thermoautotrophicum, Pyrococcus abyssi, Pyrococcus horikoshii, Thermoplasma acidophilum, Thermoplasma volcanium.

Examples of a bacterial cell include, but are not limited to those belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphlococcus, Strepromyces, Synnecoccus, and Zymomonas.

Illustrative examples of bacterial strains include but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, Staphylococcus aureus, and the like.

In general, if a bacterial host cell is used, a non-pathogenic strain is preferred. Illustrative examples of non-pathogenic strains include but are not limited to: Bacillus subtilis, Escherichia coli, Lactibacillus acidophilus, Lactobacillus helveticus, Pseudomonas aeruginosa, Pseudomonas mevalonii, Pseudomonas pudita, Rhodobacter sphaeroides, Rodobacter capsulatus, Rhodospirillum rubrum, and the like.

Illustrative examples of eukaryotic strains include but are not limited to: Aspergillus nidulans, Aspergillus niger, Aspergillus oryzae, Candida albicans, Chrysosporium lucknowense, Fusarium graminearum, Fusarium venenatum, Kluyveromyces lactis, Neurospora crassa, Pichia angusta, Pichia finlandica, Pichia kodamae, Pichia membranaefaciens, Pichia methanolica, Pichia opuntiae, Pichia pastoris, Pichia pijperi, Pichia quercuum, Pichia salictaria, Pichia thermotolerans, Pichia trehalophila, Pichia stipitis, Streptomyces ambofaciens, Streptomyces aureofaciens, Streptomyces aureus, Saccaromyces bayanus, Saccaromyces boulardi, Saccharomyces cerevisiae, Streptomyces fungicidicus, Streptomyces griseochromogenes, Streptomyces griseus, Streptomyces lividans, Streptomyces olivogriseus, Streptomyces rameus, Streptomyces tanashiensis, Streptomyces vinaceus, Trichoderma reesei and Xanthophyllomyces dendrorhous (formerly Phaffia rhodozyma).

In general, if a eukaryotic cell is used, a non-pathogenic strain is preferred. Illustrative examples of non-pathogenic strains include but are not limited to: Fusarium graminearum, Fusarium venenatum, Pichia pastoris, Saccaromyces boulardi, and Saccaromyces cerevisiae.

Examples of eukaryotic cells include but are not limited to fungal cells. Examples of fungal cell include, but are not limited to those belonging to the genera: Aspergillus, Candida, Chrysosporium, Cryotococcus, Fusarium, Kluyveromyces, Neotyphodium, Neurospora, Penicillium, Pichia, Saccharomyces, Trichoderma and Xanthophyllomyces (formerly Phaffia).

In addition, certain strains have been designated by the Food and Drug Administration as GRAS or Generally Regarded As Safe. These strains include: Bacillus subtilis, Lactibacillus acidophilus, Lactobacillus helveticus, and Saccharomyces cerevisiae.

### Isoprenoids of the Present Invention

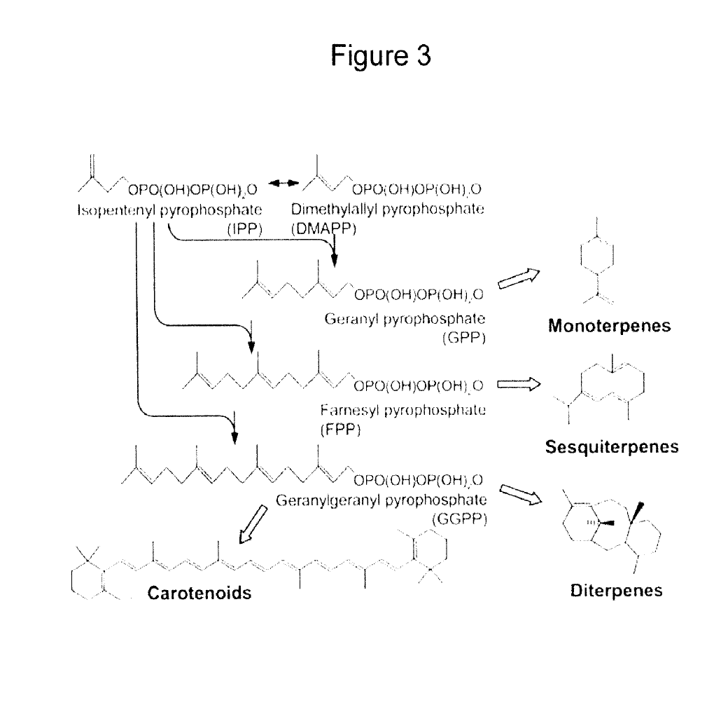

The compositions and methods of the present invention can be employed to produce a wide variety of isoprenoids, including, without limitation, any C₅ through C₂₀ or higher carbon number isoprenoids (see e.g. FIG. 3 which illustrates the conversion of IPP and DMAPP into GPP, FPP, and GGPP to make exemplary isoprenoid products). The following describes, without limitation, additional exemplary isoprenoids of the invention.

### C₅ Compounds

C₅ compounds of the invention generally are derived from IPP or DMAPP. These compounds are also known as hemiterpenes because they are derived from a single isoprene unit (IPP or DMAPP).

Isoprene

Isoprene, whose structure is

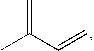

is found in many plants. Isoprene is made from IPP by isoprene synthase. Illustrative examples of suitable nucleotide sequences include but are not limited to: (AB198190; Populus alba) and (AJ294819; Polulus alba×Polulus tremula).

### C₁₀ Compounds

C₁₀ compounds of the invention generally derived from geranyl pyrophosphate (GPP) which is made by the condensation of IPP with DMAPP. An enzyme known to catalyze this step is, for example, geranyl pyrophosphate synthase. These C₁₀ compounds are also known as monoterpenes because they are derived from two isoprene units. In certain embodiments, the host cells of the present invention comprises a heterologous nucleic acid sequence that encodes an enzyme that converts IPP and DMAPP into GPP.

FIG. 3 shows schematically how IPP and DMAPP can produce GPP, which can be further processed to a monoterpene.

Illustrative examples of nucleotide sequences for geranyl pyrophosphate synthase include but are not limited to: (AF513111; Abies grandis), (AF513112; Abies grandis), (AF513113; Abies grandis), (AY534686; Antirrhinum majus), (AY534687; Antirrhinum majus), (Y17376; Arabidopsis thaliana), (AE016877, Locus AP11092; Bacillus cereus; ATCC 14579), (AJ243739; Citrus sinensis), (AY534745; Clarkia breweri), (AY953508; Ips pini), (DQ286930; Lycopersicon esculentum), (AF182828; Mentha×Piperita), (AF 182827; Mentha×piperita), (MPI249453; Mentha×piperita), (PZE431697, Locus CAD24425; Paracoccus zeaxanthinifaciens), (AY866498; Picrorhiza kurrooa), (AY351862; Vitis vinifera), and (AF203881, Locus AAF12843; Zymomonas mobilis).

GPP is then subsequently converted to a variety of C₁₀ compounds. Illustrative examples of C₁₀ compounds include but are not limited:

Carene

Carene, whose structure is

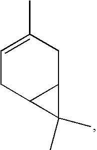

is found in the resin of many trees, particularly pine trees. Carene is made from GPP from carene synthase. Illustrative examples of suitable nucleotide sequences include but are not limited to: (AF461-460, REGION 43.1926; Picea abies) and (AF527416, REGION: 78.1871; Salvia stenophylla).

Geraniol

Geraniol (also known as rhodnol), whose structure is

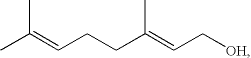

is the main component of oil-of-rose and palmarosa oil. It also occurs in geranium, lemon, and citronella. Geraniol is made from GPP by geraniol synthase. Illustrative examples of suitable nucleotide sequences include but are not limited to: (A1457070; Cinnamomum tenuipilum), (AY362553; Ocimum basilicum), (DQ234300; Perilla frutescens strain 1864), (DQ234299; Perilla citriodora strain 1861), (DQ234298; Perilla citriodora strain 4935), and (DQ088667; Perilla citriodora)

Linalool

Linalool, whose structure is

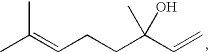

is found in many flowers and spice plants such as coriander seeds. Linalool is made from GPP by linalool synthase. Illustrative examples of a suitable nucleotide sequence include but are not limited to: (AF497-485; Arabidopsis thaliana), (AC002294, Locus AAB71482; Arabidopsis thaliana), (AY059757; Arabidopsis thaliana), (NM_{—}104793; Arabidopsis thaliana), (AF154124; Artemisia annua), (AF067603; Clarkia breweri), (AF067602; Clarkia concinna), (AF067601; Clarkia breweri), (U58314; Clarkia breweri), (AY840091; Lycopersicon esculentum), (DQ263741; Lavandula angustifolia), (AY083653; Mentha citrate), (AY693647; Ocimum basilicum), (XM_{—}463918; Oryza sativa), (AP004078, Locus BAD07605; Oryza sativa), (XM_{—}463918, Locus XP_{—}463918; Oryza sativa), (AY917193; Perilla citriodora), (AF271259; Perilla frutescens), (AY473623; Picea abies), (DQ195274; Picea sitchensis), and (AF444798; Perilla frutescens var. crispa cultivar No. 79).

Limonene

Limonene, whose structure is

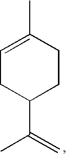

is found in the rind of citrus fruits and peppermint. Limonene is made from GPP by limonene synthase. Illustrative examples of suitable nucleotide sequences include but are not limited to: (+)-limonene synthases (AF514287, REGION: 47.1867; Citrus limon) and (AY055214, REGION: 48.1889; Agastache rugosa) and (−)-limonene synthases (DQ195275, REGION: 1.1905; Picea sitchensis), (AF006193, REGION: 73.1986; Abies grandis), and (MHC4SLSP, REGION: 29.1828; Mentha spicata).

Myrcene

Myrcene, whose structure is

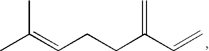

is found in the essential oil in many plants including bay, verbena, and myrcia from which it gets its name. Myrcene is made from GPP by myrcene synthase. Illustrative examples of suitable nucleotide sequences include but are not limited to: (U87908; Abies grandis), (AY195609; Antirrhinum majus), (AY195608; Antirrhinum majus), (NM_{—}127982; Arabidopsis thaliana TPS10), (NM_{—}113485; Arabidopsis thaliana ATTPS-CIN), (NM_{—}113483; Arabidopsis thaliana ATTPS-CIN), (AF271259; Perilla frutescens), (AY473626; Picea abies), (AF369919; Picea abies), and (AJ304839; Quercus ilex).

Ocimene

α- and β-Ocimene, whose structures are

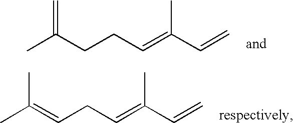

are found in a variety of plants and fruits including Ocimum basilicum and is made from GPP by ocimene synthase. Illustrative examples of suitable nucleotide sequences include but are not limited to: (AY195607; Antirrhinum majus), (AY195609; Antirrhinum majus), (AY195608; Antirrhinum majus), (AK221024; Arabidopsis thaliana), (NM_{—}113485; Arabidopsis thaliana ATTPS-CIN), (NM_{—}113483; Arabidopsis thaliana ATTPS-CIN), (NM_{—}117775; Arabidopsis thaliana ATTPS03), (NM_{—}001036574; Arabidopsis thaliana ATTPS03), (NM_{—}127982; Arabidopsis thaliana TPS10), (AB110642; Citrus unshiu CitMTSL4), and (AY575970; Lotus corniculatus var. japonicus).

α-Pinene

α-Pinene, whose structure is

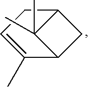

is found in pine trees and eucalyptus. α-Pinene is made from GPP by α-pinene synthase. Illustrative examples of suitable nucleotide sequences include but are not limited to: (+) α-pinene synthase (AF543530, REGION: 1.1887; Pinus taeda), (−) α-pinene synthase (AF543527, REGION: 32.1921; Pinus taeda), and (+)/(−) α-pinene synthase (AGU87909, REGION: 6111892; Abies grandis).

β-Pinene

β-Pinene, whose structure is

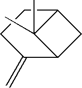

is found in pine trees, rosemary, parsley, dill, basil, and rose. β-Pinene is made from GPP by β-pinene synthase. Illustrative examples of suitable nucleotide sequences include but are not limited to: (−) β-pinene synthases (AF276072, REGION: 1.1749; Artemisia annua) and (AF514288, REGION: 26.1834; Citrus limon).

Sabinene

Sabinene, whose structure is

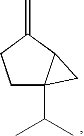

is found in black pepper, carrot seed, sage, and tea trees. Sabinene is made from GPP by sabinene synthase. An illustrative example of a suitable nucleotide sequence includes but is not limited to AF051901, REGION: 26.1798 from Salvia officinalis.

### γ-Terpinene

γ-Terpinene, whose structure is

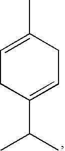

is a constituent of the essential oil from citrus fruits. Biochemically, γ-terpinene is made from GPP by a γ-terpinene synthase. Illustrative examples of suitable nucleotide sequences include: (AF514286, REGION: 30.1832 from Citrus limon) and (AB110640, REGION 1.1803 from Citrus unshiu).

Terpinolene

Terpinolene, whose structure is

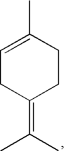

is found in black currant, cypress, guava, lychee, papaya, pine, and tea. Terpinolene is made from GPP by terpinolene synthase. Illustrative example of a suitable nucleotide sequences include but is not limited to (AY693650 from Oscimum basilicum) and (AY906866, REGION: 10.1887 from Pseudotsuga menziesii).

### C₁₅ Compounds

C₁₅ compounds of the invention generally derive from farnesyl pyrophosphate (FPP) which is made by the condensation of two molecules of IPP with one molecule of DMAPP. An enzyme known to catalyze this step is, for example, farnesyl pyrophosphate synthase. These C₁₅ compounds are also known as sesquiterpenes because they are derived from three isoprene units. In certain embodiments, the host cells of the present invention comprises a heterologous nucleic acid sequence that encodes an enzyme that converts IPP and DMAPP into FPP.

FIG. 3 shows schematically how IPP and DMAPP can be combined to produce FPP, which can be further processed to a sesquiterpene.

Illustrative examples of nucleotide sequences which encode farnesyl pyrophosphate include but are not limited to: (AF461050; Bos taurus), (AB003187, Micrococcus luteus), (AE009951, Locus AAL95523; Fusobacterium nucleatum subsp. nucleatum ATCC 25586), (GFFPPSGEN; Gibberella fujikuroi), (AB016094, Synechococcus elongatus), (CP000009, Locus AAW60034; Gluconobacter oxydans 621H), (AF019892; Helianthus annuus), (HUMFAPS; Homo sapiens), (KLPFPSQCR; Kluyveromyces lactis), (LAU15777; Lupinus albus), (LAU20771; Lupinus albus), (AF309508; Mus musculus), (NCFPPSGEN; Neurospora crassa), (PAFPS1; Parthenium argentatum), (PAFPS2; Parthenium argentatum), (RATFAPS; Rattus norvegicus), (YSCFPP; Saccharomyces cerevisiae), (D89104; Schizosaccharomyces pombe), (CP000003, Locus AAT87386; Streptococcus pyogenes), (CP000017, Locus AAZ51849; Streptococcus pyogenes), (NC_{—}008022, Locus YP_{—}598856; Streptococcus pyogenes MGAS10270), (NC_{—}008023, Locus YP_{—}600845; Streptococcus pyogenes MGAS2096), (NC_{—}008024, Locus YP_{—}602832; Streptococcus pyogenes MGAS10750), and (MZEFPS; Zea mays, (AB021747, Oryza sativa FPPS1 gene for farnesyl diphosphate synthase), (AB028044, Rhodobacter sphaeroides), (AB028046, Rhodobacter capsulatus), (AB028047, Rhodovulum sulfidophilum), (AAU36376; Artemisia annua), (AF112881 and AF136602, Artemisia annua), (AF384040, Mentha×piperita), (D00694, Escherichia coli K-12), (D13293, B. stearothermophilus), (D85317, Oryza sativa), (ATU80605; Arabidopsis thaliana), (ATHFPS2R; Arabidopsis thaliana), (X75789, A. thaliana), (Y12072, G. arboreum), (Z49786, H. brasiliensis), (U80605, Arabidopsis thaliana farnesyl diphosphate synthase precursor (FPS1) mRNA, complete cds), (X76026, K lactis FPS gene for farnesyl diphosphate synthetase, QCR8 gene for bc1 complex, subunit VIII), (X82542, P. argentatum mRNA for farnesyl diphosphate synthase (FPS1), (X82543, P. argentatum mRNA for farnesyl diphosphate synthase (FPS2), (BC010004, Homo sapiens, farnesyl diphosphate synthase (farnesyl pyrophosphate synthetase, dimethylallyltranstransferase, geranyltranstransferase), clone MGC 15352 IMAGE, 4132071, mRNA, complete cds) (AF234168, Dictyostelium discoideum farnesyl diphosphate synthase (Dfps), (L46349, Arabidopsis thaliana farnesyl diphosphate synthase (FPS2) mRNA, complete cds), (L46350, Arabidopsis thaliana farnesyl diphosphate synthase (FPS2) gene, complete cds), (L46367, Arabidopsis thaliana farnesyl diphosphate synthase (FPS1) gene, alternative products, complete cds), (M89945, Rat farnesyl diphosphate synthase gene, exons 1-8), (NM_{—}002004, Homo sapiens farnesyl diphosphate synthase (farnesyl pyrophosphate synthetase, dimethylallyltranstransferase-, geranyltranstransferase) (FDPS), mRNA), (U36376, Artemisia annua farnesyl diphosphate synthase (fps1) mRNA, complete cds), (XM_{—}001352, Homo sapiens farnesyl diphosphate synthase (farnesyl pyrophosphate synthetase, dimethylallyltranstransferase-, geranyltranstransferase) (FDPS), mRNA), (XM_{—}034497, Homo sapiens farnesyl diphosphate synthase (farnesyl pyrophosphate synthetase, dimethylallyltranstransferase, geranyltranstransferase) (FDPS), mRNA), (XM_{—}034498, Homo sapiens farnesyl diphosphate synthase (farnesyl pyrophosphate synthetase, dimethylallyltranstransferase, geranyltranstransferase) (FDPS), mRNA), (XM_{—}034499, Homo sapiens farnesyl diphosphate synthase (farnesyl pyrophosphate synthetase, dimethylallyltranstransferase, geranyltranstransferase) (FDPS), mRNA), and (XM_{—}0345002, Homo sapiens farnesyl diphosphate synthase (farnesyl pyrophosphate synthetase, dimethylallyltranstransferase, geranyltranstransferase) (FDPS), mRNA).

Alternatively, FPP can also be made by adding IPP to GPP. Illustrative examples of nucleotide sequences encoding for an enzyme capable of this reaction include but are not limited to: (AE000657, Locus AAC06913; Aquifex aeolicus VF5), (NM_{—}202836; Arabidopsis thaliana), (D84432, Locus BAA12575; Bacillus subtilis), (U12678, Locus AAC28894; Bradyrhizobium japonicum USDA 110), (BACFDPS; Geobacillus stearothermophilus), (NC_{—}002940, Locus NP_{—}873754; Haemophilus ducreyi 35000HP), (L42023, Locus AAC23087; Haemophilus influenzae Rd KW20), (J05262; Homo sapiens), (YP_{—}395294; Lactobacillus sakei subsp. sakei 23K), (NC_{—}005823, Locus YP_{—}000273; Leptospira interrogans serovar Copenhageni str. Fiocruz L1-130), (AB003187; Micrococcus luteus), (NC_{—}002946, Locus YP_{—}208768; Neisseria gonorrhoeae FA 1090), (U00090, Locus AAB91752; Rhizobium sp. NGR234), (J05091; Saccharomyces cerevisae), (CP000031, Locus AAV93568; Silicibacter pomeroyi DSS-3), (AE008481, Locus AAK99890; Streptococcus pneumoniae R6), and (NC_{—}004556, Locus NP 779706; Xylella fastidiosa Temecula1).

FPP is then subsequently converted to a variety of C₁₅ compounds. Illustrative examples of C₁₅ compounds include but are not limited to:

Amorphadiene

Amorphadiene, whose structure is

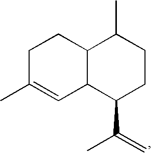

is a precursor to artemisinin which is made by Artemisia anna. Amorphadiene is made from FPP by amorphadiene synthase. An illustrative example of a suitable nucleotide sequence is SEQ ID NO. 37 of U.S. Patent Publication No. 2004/0005678.

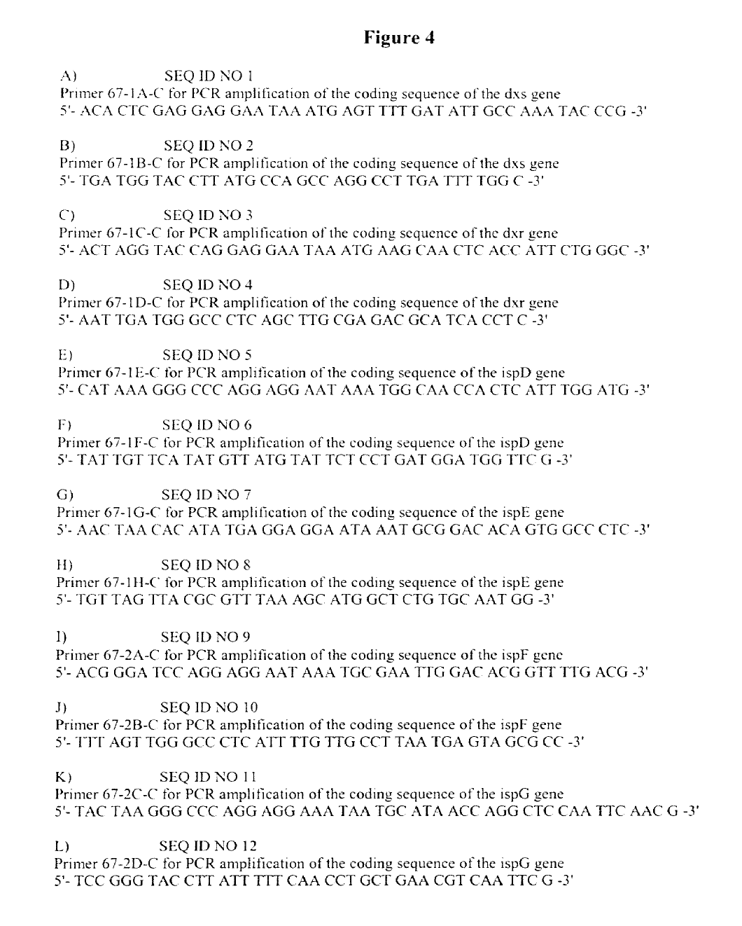

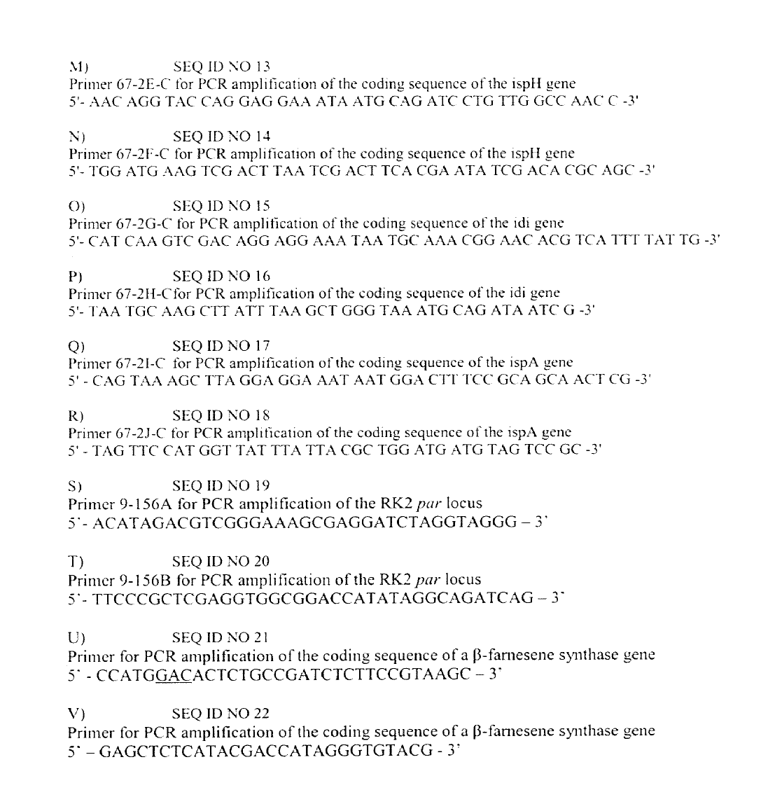

FIG. 4 shows schematically how IPP and DMAPP can be combined to produce FPP, which can then be further processed to produce amophadiene.

α-Farnesene

α-Farnesene, whose structure is

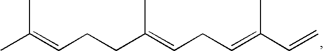

is found in various biological sources including but not limited to the Dufour's gland in ants and in the coating of apple and pear peels. α-Farnesene is made from FPP by α-farnesene synthase. Illustrative examples of suitable nucleotide sequences include but are not limited to DQ309034 from Pyrus communis cultivar d'Anjou (pear; gene name AFS1) and AY182241 from Malus domestica (apple; gene AFS1). Pechouus et al., Planta 219(1):84-94 (2004).

β-Farnesene

β-Farnesene, whose structure is

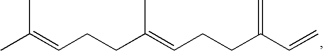

is found in various biological sources including but not limited to aphids and essential oils such as from peppermint. In some plants such as wild potato, β-farnesene is synthesized as a natural insect repellent. β-Farnesene is made from FPP by β-farnesene synthase. Illustrative examples of suitable nucleotide sequences include but is not limited to GenBank accession number AF024615 from Mentha×piperita (peppermint; gene Tspa11), and AY835398 from Artemisia annua. Picaud et al., Phytochemistry 66(9): 961-967 (2005).

Farnesol

Farnesol, whose structure is

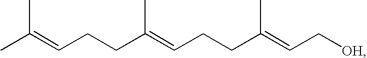

is found in various biological sources including insects and essential oils such as from cintronella, neroli, cyclamen, lemon grass, tuberose, and rose. Farnesol is made from FPP by a hydroxylase such as farnesol synthase. Illustrative examples of suitable nucleotide sequences include but are not limited to GenBank accession number AF529266 from Zea mays and YDR481C from Saccharomyces cerevisiae (gene Pho8). Song, L., Applied Biochemistry and Biotechnology 128:149-158 (2006).

Nerolidol

Nerolidol, whose structure is

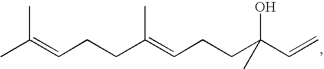

is also known as peruviol, and is found in various biological sources including as essential oils such as from neroli, ginger, jasmine, lavender, tea tree, and lemon grass. Nerolidol is made from FPP by a hydroxylase such as nerolidol synthase. An illustrative example of a suitable nucleotide sequence includes but is not limited to AF529266 from Zea mays (maize; gene tps1).

Patchoulol

Patchoulol, whose structure is

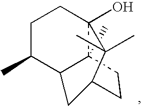

is also known as patchouli alcohol and is a constituent of the essential oil of Pogostemon patchouli. Patchouliol is made from FPP by patchouliol synthase. An illustrative example of a suitable nucleotide sequence includes but is not limited to AY508730 REGION: 1.1659 from Pogostemon cablin.

Valencene

Valencene, whose structure is

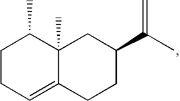

is one of the main chemical components of the smell and flavour of oranges and is found in orange peels. Valencene is made from FPP by nootkatone synthase. Illustrative examples of a suitable nucleotide sequence includes but is not limited to AF441124 REGION: 1.1647 from Citrus sinensis and AY917195 REGION: 1.1653 from Perilla frutescens.

### C₂₀ Compounds

C₂₀ compounds of the invention generally derived from geranylgeraniol pyrophosphate (GGPP) which is made by the condensation of three molecules of IPP with one molecule of DMAPP. An enzyme known to catalyze this step is, for example, geranylgeranyl pyrophosphate synthase. These C₂₀ compounds are also known as diterpenes because they are derived from four isoprene units. In certain embodiments, the host cells of the present invention comprises a heterologous nucleic acid sequence that encodes an enzyme that converts IPP and DMAPP into GGPP.

FIG. 3 shows schematically how IPP and DMAPP can be combined to produce GGPP, which can be further processed to a diterpene, or can be further processed to produce a carotenoid.

Illustrative examples of nucleotide sequences for geranylgeranyl pyrophosphate synthase include but are not limited to: (ATHGERPYRS; Arabidopsis thaliana), (BT005328; Arabidopsis thaliana), (NM 119845; Arabidopsis thaliana), (NZ_AAJM01000380, Locus ZP_{—}00743052; Bacillus thuringiensis serovar israelensis, ATCC 35646 sq1563), (CRGGPPS; Catharanthus roseus), (NZ_AABF02000074, Locus ZP_{—}00144509; Fusobacterium nucleatum subsp. vincentii, ATCC 49256), (GFGGPPSGN; Gibberella fujikuroi), (AY371321; Ginkgo biloba), (AB055496; Hevea brasiliensis), (AB017971; Homo sapiens), (MCI276129; Mucor circinelloides f. lusitanicus), (AB016044; Mus musculus), (AABX01000298, Locus NCU01427; Neurospora crassa), (NCU20940; Neurospora crassa), (NZ_AAKL01000008, Locus ZP_{—}00943566; Ralstonia solanacearum UW551), (AB118238; Rattus norvegicus), (SCU31632; Saccharomyces cerevisiae), (AB016095; Synechococcus elongates), (SAGGPS; Sinapis alba), (SSOGDS; Sulfolobus acidocaldarius), (NC_{—}007759, Locus YP_{—}461832; Syntrophus aciditrophicus SB), and (NC_{—}006840, Locus YP_{—}204095; Vibrio fischeri ES114).

Alternatively, GGPP can also be made by adding IPP to FPP. Illustrative examples of nucleotide sequences encoding an enzyme capable of this reaction include but are not limited to: (NM_{—}112315; Arabidopsis thaliana), (ERWCRTE; Pantoea agglomerans), (D90087, Locus BAA14124; Pantoea ananatis), (X52291, Locus CAA36538; Rhodobacter capsulatus), (AF195122, Locus AAF24294; Rhodobacter sphaeroides), and (NC_{—}004350, Locus NP_{—}721015; Streptococcus mutans UA 159).

GGPP is then subsequently converted to a variety of C₂₀ isoprenoids. Illustrative examples of C₂₀ compounds include but are not limited to:

Geranylgeraniol

Geranylgeraniol, whose structure is

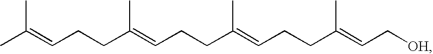

is a constituent of wood oil from Cedrela toona and of linseed oil. Geranylgeraniol can be made by e.g., adding to the expression constructs a phosphatase gene after the gene for a GGPP synthase.

Abietadiene

Abietadiene encompasses the following isomers:

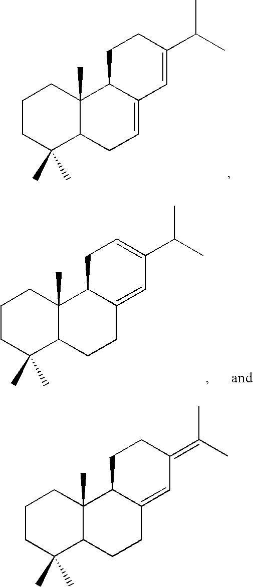

and is found in trees such as Abies grandis. Abietadiene is made by abietadiene synthase. An illustrative example of a suitable nucleotide sequence includes but are not limited to: (U50768; Abies grandis) and (AY473621; Picea abies).

### C₂₀₊ Compounds

C₂₀₊ compounds are also within the scope of the present invention. Illustrative examples of such compounds include sesterterpenes (C₂₅ compound made from five isoprene units), triterpenes (C₃₀ compounds made from six isoprene units), and tetraterpenes (C₄₀ compound made from eight isoprene units). These compounds are made by using similar methods described herein and substituting or adding nucleotide sequences for the appropriate synthase(s).

Although the invention has been described in conjunction with specific embodiments thereof, the foregoing description is intended to illustrate and not limit the scope of the invention. Other aspects, advantages, and modifications within the scope of the invention will be apparent to those skilled in the art to which the invention pertains. All patents, patent applications, and publications mentioned herein are hereby incorporated by reference in their entireties.

### EXAMPLES

The practice of the present invention can employ, unless otherwise indicated, conventional techniques of the biosynthetic industry and the like, which are within the skill of the art. To the extent such techniques are not described fully herein, one can find ample reference to them in the scientific literature.

In the following examples, efforts have been made to ensure accuracy with respect to numbers used (for example, amounts, temperature, and so on), but variation and deviation can be accommodated, and in the event a clerical error in the numbers reported herein exists, one of ordinary skill in the arts to which this invention pertains can deduce the correct amount in view of the remaining disclosure herein. Unless indicated otherwise, temperature is reported in degrees Celsius, and pressure is at or near atmospheric pressure at sea level. All reagents, unless otherwise indicated, were obtained commercially. The following examples are intended for illustrative purposes only and do not limit in any way the scope of the present invention.

### Example 1

This example describes methods for making expression plasmids that encode enzymes of the DXP pathway organized in operons.

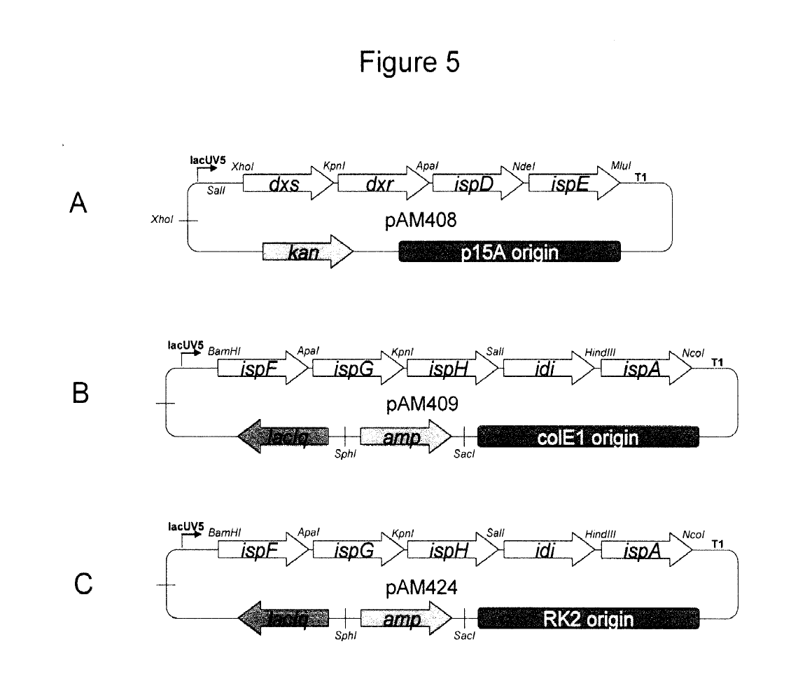

Expression plasmid pAM408 was generated by inserting genes encoding enzymes of the “top” DXP pathway into the pAM29 vector. Vector pAM29 was created by assembling the p15A origin of replication and kan resistance gene from plasmid pZS24-MCS1 (Lutz and Bujard Nucl Acids Res. 25:1203-1210 (1997)) with an oligonucleotide-generated lacUV5 promoter. Enzymes of the “top” DXP pathway include 1-deoxy-D-xylulose-5-phosphate synthase (encoded by the dxs gene of Escherichia coli), 1-deoxy-D-xylulose-5-phosphate reductoisomerase (encoded by the dxr gene of Escherichia coli), 4-diphosphocytidyl-2C-methyl-D-erythritol synthase (encoded by the ispD gene of Escherichia coli), and 4-diphosphocytidyl-2C-methyl-D-erythritol synthase (encoded by the ispE gene of Escherichia coli), which together transform pyruvate and D-glyceraldehyde-3-phosphate to 4-diphosphocytidyl-2C-methyl-D-erythritol-2-phosphate. An operon encoding enzymes of the “top” DXP pathway was generated by PCR amplifying the dxs (GenBank accession number U00096 REGION: 437539.439401), dxr (GenBank accession number U00096 REGION: 193521.194717), ispD (GenBank accession number U00096 REGION: 2869803.2870512), and ispE (GenBank accession number U00096 REGION 1261249.1262100) genes from Escherichia coli strain DH1 (ATCC #33849) with added optimal Shine Dalgamo sequences and 5′ and 3′ restriction enzyme sites using the PCR primers shown in FIGS. 4A-H. The PCR products were resolved by gel electrophoresis, gel extracted using a Qiagen (Valencia, Calif.) gel purification kit, digested to completion using appropriate restriction enzymes (XhoI and KpnI for the PCR product comprising the dxs gene; KpnI and ApaI for the PCR product comprising the dxr gene; ApaI and NdeI for the PCR product comprising the ispD gene; NdeI and MluI for the PCR product comprising the ispE gene), and purified using a Qiagen (Valencia, Calif.) PCR purification kit. Roughly equimolar amounts of each PCR product were then added to a ligation reaction to assemble the individual genes into an operon. From this ligation reaction, 1 μl of reaction mixture was used to PCR amplify 2 separate gene cassettes, namely the dxs-dxr and the ispD-ispE gene cassettes. The dxs-dxr gene cassette was PCR amplified using primers 67-1A-C and 67-1D-C (FIGS. 4A and 4D, respectively), and the ispD-ispE gene cassette was PCR amplified using primers 67-1E-C and 67-1H-C (FIGS. 4E and 4H, respectively). The two PCR products were resolved by gel electrophoresis, and gel extracted. The PCR product comprising the dxs-dxr gene cassette was digested to completion using XhoI and ApaI restriction enzymes, the PCR product comprising the ispD-ispE gene cassette was digested to completion using ApaI and MluI restriction enzymes, the two PCR products were purified, and the isolated DNA fragments were inserted into the SalI MluI restriction enzyme site of the pAM29 vector, yielding expression plasmid pAM408 (see FIG. 5A for a plasmid map).

Expression plasmid pAM409 was generated by inserting genes encoding enzymes of the “bottom” DXP pathway into the pAM369 vector. Vector pAM369 was created by assembling the p15A origin of replication from pAM29 and beta-lactamase gene for ampicillin resistance from pZE12-luc (Lutz and Bujard Nucl Acids Res. 25:1203-1210 (1997)) with an oligonucleotide-generated lacUV5 promoter. Enzymes of the “bottom” DXP pathway include 2C-methyl-D-erythritol 2,4-cyclodiphosphate synthase (encoded by the ispF gene of Escherichia coli), 1-hydroxy-2-methyl-2-(E)-butenyl-4-diphosphate synthase (encoded by the ispG gene of Escherichia coli), and isopentenyl/dimethylallyl diphosphate synthase (encoded by the ispH gene of Escherichia coli), which together transform 4-diphosphocytidyl-2C-methyl-D-erythritol-2-phosphate to IPP and DMAPP. IPP is also converted to DMAPP through the activity of isopentyl diphosphate isomerase (encoded by the idi gene of Escherichia coli). DMAPP can be further converted to FPP through the activity of farnesyl diphosphate synthase (encoded by the ispA gene of Escherichia coli). An operon encoding enzymes of the “bottom” DXP pathway as well as an isopentyl diphosphate isomerase and a farnesyl diphosphate synthase was generated by PCR amplifying the ispF (GenBank accession number U00096 REGION: 2869323.2869802), ispG (GenBank accession number U00096 REGION: 2638708.2639826), ispH (GenBank accession number U00096 REGION: 26277.27227), idi (GenBank accession number AF119715), and ispA (GenBank accession number D00694 REGION: 484.1383) genes from Escherichia coli strain DH1 (ATCC #33849) with added optimal Shine Dalgarno sequences and 5′ and 3′ restriction enzyme sites using the PCR primers shown in FIGS. 4I-R. The PCR products were resolved by gel electrophoresis, gel extracted, digested with the appropriate restriction enzymes (BamHI and ApaI for the PCR product comprising the ispF gene; KpnI and ApaI for the PCR product comprising the ispG gene; SalI and KpnI for the PCR product comprising the ispH gene; SalI and HindIII for the PCR product comprising the idi gene; HindIII and NcoI for the PCR product comprising the ispA gene), and purified. Roughly equimolar amounts of each PCR product were then added to a ligation reaction to assemble the individual genes into an operon. From this ligation reaction, 1 μl of reaction mixture was used to PCR amplify 2 separate gene cassettes, namely the ispF-ispG and the ispH-idi-ispA gene cassettes. The ispF-ispG gene cassette was PCR amplified using primers 67-2A-C and 67-2D-C (FIGS. 4I and 4L, respectively), and the ispH-idi-ispA gene cassette was PCR amplified using primers 67-2E-C and 67-2J-C (FIGS. 4M and 4R, respectively). The two PCR products were resolved by gel electrophoresis, and gel extracted. The PCR product comprising the ispF-ispG gene cassette was digested to completion using BamHI and KpnI restriction enzymes, the PCR product comprising the ispH-idi-ispA gene cassette was digested to completion using KpnI and NcoI restriction enzymes, the two PCR products were purified, and the two isolated DNA fragments were inserted into the BamHI NcoI restriction enzyme site of the pAM369 vector, yielding expression plasmid pAM409 (see FIG. 5B for a plasmid map).

Expression plasmid pAM424, a derivative of expression plasmid pAM409 containing the broad-host range

RK2 origin of replication, was generated by transferring the lacUV5 promoter and the ispFGH-idi-ispA operon of pAM409 to the pAM257 vector. Vector pAM257 was generated as follows: the RK2 par locus was PCR-amplified from RK2 plasmid DNA (Meyer et al. (1975) Science 190:1226-1228) using primers 9-156A (FIG. 4S) and 9-156B (FIG. 4T), the 2.6 kb PCR product was digested to completion using AatII and XhoI restriction enzymes, and the DNA fragment was inserted into a plasmid containing the p15 origin of replication and the chloramphenicol resistance gene from vector pZA31-luc (Lutz and Bujard (1997) Nucl Acids Res. 25:1203-1210.), yielding plasmid pAM37-par; pAM37-par was digested to completion using restriction enzymes SacI and HindIII, the reaction mixture was resolved by gel electrophoresis, the DNA fragment comprising the RK2 par locus and the chloramphenicol resistance gene was gel extracted, and the isolated DNA fragment was inserted into the SacI HindIII site of the mini-RK2 replicon pRR10 (Roberts et al. (1990) J Bacteriol. 172:6204-6216), yielding vector pAM133; pAM133 was digested to completion using BglII and HindIII restriction enzymes, the reaction mixture was resolved by gel electrophoresis, the approximately 6.4 kb DNA fragment lacking the ampicillin resistance gene and oriT conjugative origin was gel extracted, and the isolated DNA fragment was ligated with a synthetically generated DNA fragment comprising a multiple cloning site that contained PciI and XhoI restriction enzyme sites, yielding vector pAM257. Expression plasmid pAM409 was digested to completion using XhoI and PciI restriction enzymes, the reaction mixture was resolved by gel electrophoresis, the approximately 4.4 kb DNA fragment was gel extracted, and the isolated DNA fragment was inserted into the XhoI PciI restriction enzyme site of the pAM257 vector, yielding expression plasmid pAM424 (see FIG. 5C for a plasmid map).

### Example 2

This example describes the generation of an Escherichia coli host strain for the production of amorpha-4,11-diene.

Host strain B003 was created by transforming chemically competent Escherichia coli DH10B cells with expression plasmid pAM3. Host strain B617 was created by transforming chemically competent Escherichia coli DH10B cells with expression plasmids pAM408 and pAM3. Host strain B618 was created by transforming chemically competent Escherichia coli DH10B cells with expression plasmids pAM424 and pAM3. Host strain B619 was created by transforming chemically competent Escherichia coli D1110B cells with expression plasmids pAM408, pAM424, and pAM3.

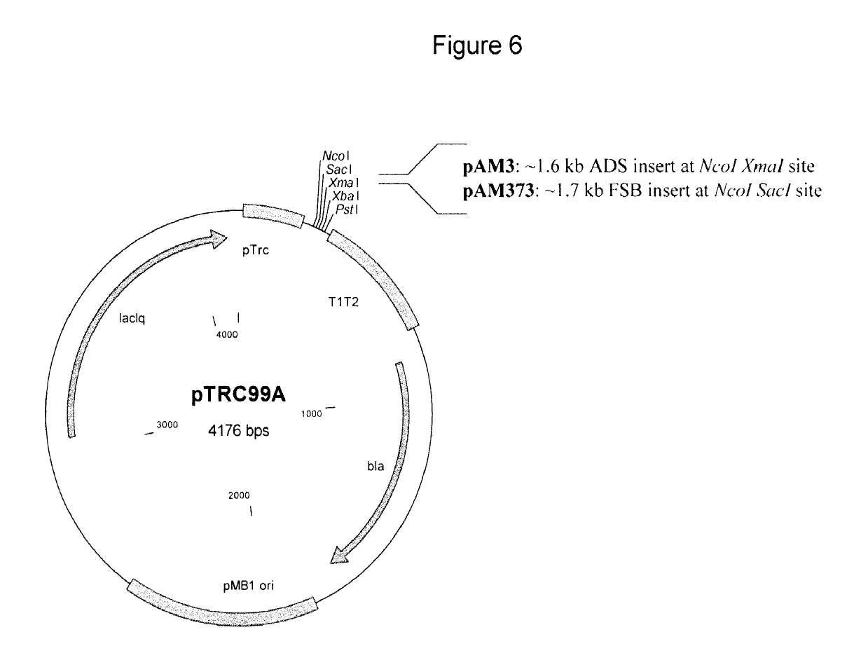

Expression plasmid pAM3 was generated by inserting a nucleotide sequence encoding an amorpha-4,11-diene synthase (“ADS”) into vector pTrc99A. The amorpha-4,11-diene synthase sequence was generated synthetically, so that upon translation the amino acid sequence would be identical to that described by Merke et al. (2000) Ach. Biochem. Biophys. 381:173-180, so that the nucleotide sequence encoding the amorpha-4,11-diene synthase was optimized for expression in Escherichia coli, and so that the nucleotide sequence was flanked by a 5′ NcoI and a 3′ XmaI restriction enzyme site (see U.S. Pat. No. 7,192,751). The nucleotide sequence was digested to completion using NcoI and XmaI restriction enzymes, the reaction mixture was resolved by gel electrophoresis, the approximately 1.6 kb DNA fragment was extracted, and the isolated DNA fragment was inserted into the NcoI and XmaI restriction enzyme site of the pTrc99A vector (Amman et al. (1985) Gene 40:183-190), yielding expression plasmid pAM3 (see FIG. 6 for a plasmid map).

B003 host cell transformants were selected on Luria-Bertani (LB) media containing 100 μg/ml carbenicillin. B617 host cell transformants were selected on LB media containing 100 ug/mL carbenicillin and 50 ug/mL kanamycin. B618 host cell transformants were selected on LB media containing 100 ug/mL carbenicillin and 35 μg/ml chloramphenicol. B619 host cell transformants were selected on LB media containing 100 μg/ml carbenicillin, 50 μg/ml kanamycin, and 35 μg/ml chloramphenicol.

Single colonies were transferred from LB agar plates containing host cell transformants to culture tubes containing 5 mL of LB liquid medium and antibiotics as detailed above. The cultures were incubated by shaking at 30° C. on a rotary shaker at 250 rpm for 30 hours, at which point cell growth was arrested by chilling the cultures on ice. The cells were stored at −80° C. in cryo-vials in 1 mL frozen aliquots made up of 400 uL ice cold sterile 50% glycerol and 600 uL liquid culture.

### Example 3

This example demonstrates the production of amorpha-4,11-diene in the Escherichia coli host strains of Example 2.

Seed flasks were grown overnight by adding the 1 mL stock aliquot to a 125 mL flask containing 25 mL M9-MOPS and antibiotics as detailed above. The cultures were used to inoculate 250 mL baffled flasks containing 40 mL M9-MOPS minimal medium, 45 ug/mL thiamine, micronutrients, 1.00E-5 mol/L FeSO₄, 0.1 M MOPS, 0.5% yeast extract, 20 g/L of D-glucose, and antibiotics at an initial OD₆₀₀ of approximately 0.05. Cultures were incubated by shaking at 30° C. in a humidified incubating shaker at 250 RPM until they reached an OD₆₀₀ of 0.2 to 0.3, at which point the production of amorphadiene in the host cells was induced with 1 mM IPTG (40 uL of 1M IPTG added to the culture medium). Cultures were overlain with 8 mL dodecane to capture the amorpha-4,11-diene. Samples were taken at various time points, and the amorpha-4,11-diene concentration in the samples, as well as the OD₆₀₀ of the cultures, were measured at each time point. Dry cell weight (DCW) was calculated according to the following verified formula: DCW=OD₆₀₀×0.4.

Amorpha-4,11-diene concentration was measured by transferring 100 uL samples of the upper dodecane layer of each flask to a clean tube, centrifuging the samples to separate out any remaining cells or media, layer-diluting 10 uL aliquots of each dodecane sample into 990 uL ethyl acetate spiked with beta- or trans-caryophyllene as an internal standard in a clean glass GC vial, vortexing the mixture for 30 seconds, and analyzing the ethyl acetate samples by gas chromatography-mass spectrometry (GC/MS). Analyses were performed on a Hewlett-Packard 6890 gas chromatograph/mass spectrometer as described in Martin et al. (2001) Biotechnol. Bioeng. 75:497-503, by scanning for molecular ions 189 m/z and 204 m/z. To expedite run times, the temperature program and column matrix were modified to achieve optimal peak resolution and the shortest overall runtime. A 1 uL sample was separated on the GC using a DB-XLB column (available from Agilent Technologies, Inc., Palo Alto, Calif.) and helium carrier gas. The oven cycle for each sample was 80° C. for 2 minutes, increasing temperature at 30° C./minute to a temperature of 160° C., increasing temperature at 3° C./minute to a temperature of 170° C., increasing temperature at 50° C./minute to 300° C., and a hold at 300° C. for 2 minutes. The resolved samples were analyzed by a Hewlett-Packard model 5973 mass-selective detector that monitored ions 189 m/z and 204 m/z. Previous mass spectra demonstrated that the amorpha-4,11-diene synthase product was amorpha-4,11-diene, and that amorpha-4,11-diene had a retention time of 3.48 minutes using this GC protocol. Amorpha-4,11-diene titers were calculated by comparing generated peak areas to a quantitative calibration curve of purified amorpha-4,11-diene in caryophyllene-spiked ethyl acetate Experiments were performed using 2 independent clones of each host strain and results were averaged. Deviation between samples was less than 10%.

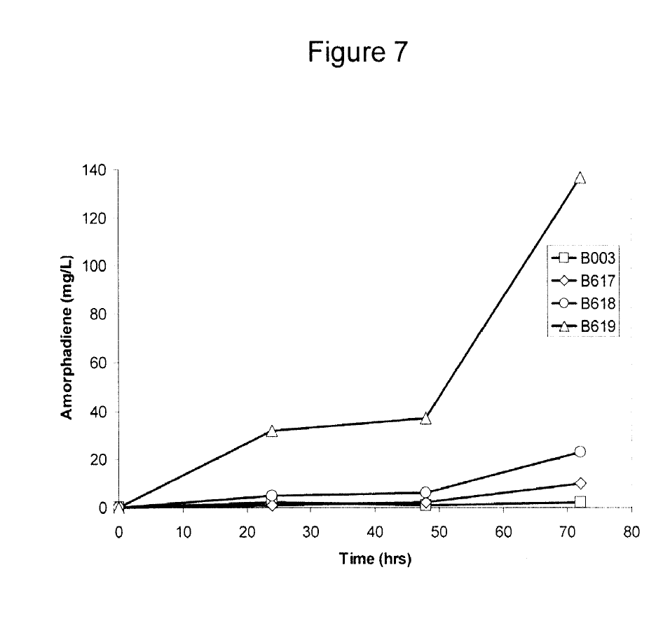

As shown in FIG. 7, Escherichia coli host strain B619, which comprises nucleotide sequences encoding enzymes of the full engineered DXP pathway, produced approximately 45 mg/g DCW amorpha-4,11-diene.

### Example 4

This example describes the generation of an Escherichia coli host strains for the production of □-farnesene.

Host strain B650 was created by transforming chemically competent Escherichia coli DH10B cells with expression plasmid pAM373. Host strain B00651 was created by transforming chemically competent Escherichia coli DH10B cells with expression plasmids pAM408 and pAM373. Host strain B652 was created by transforming chemically competent Escherichia coli DH10B cells with expression plasmids pAM424 and pAM373. Host strain B653 was created by transforming chemically competent Escherichia coli DH10B cells with expression plasmids pAM408, pAM424, and pAM373.

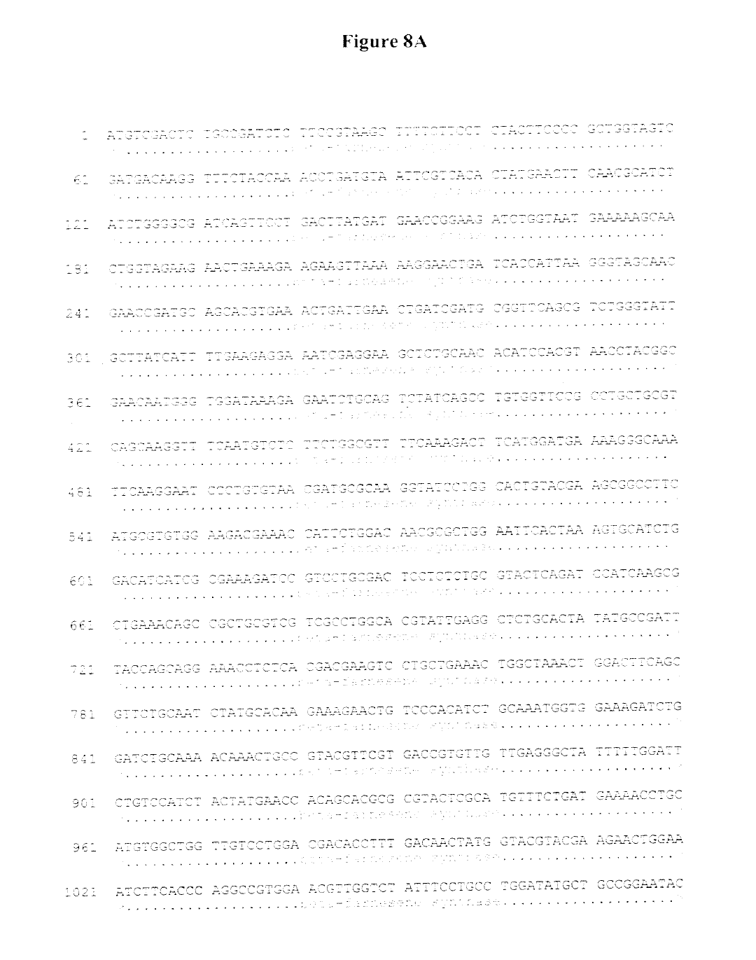

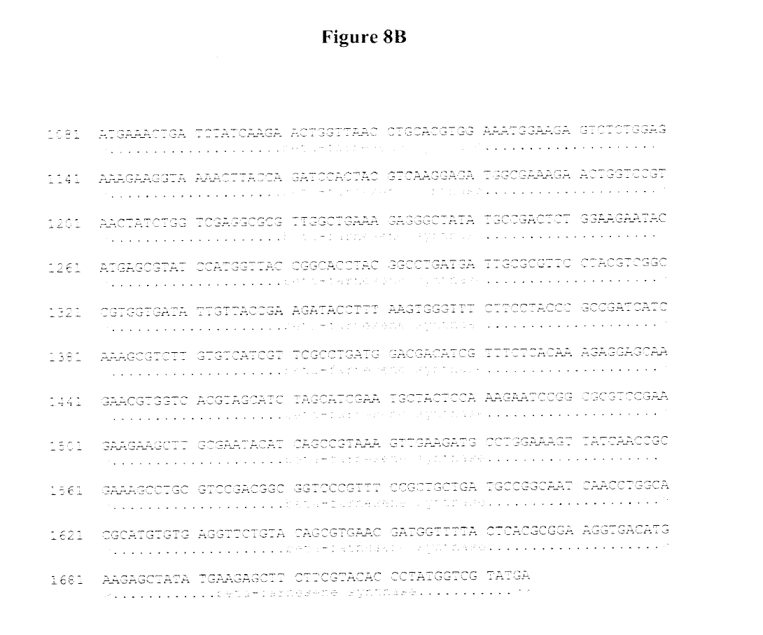

Expression plasmid pAM373 was generated by inserting a nucleotide sequence encoding the β-farnesene synthase (“FSB”) of Artemisia annua (GenBank accession number AY835398), codon-optimized for expression in Escherichia coli, into the pTrc99A vector. The nucleotide sequence encoding the β-farnesene synthase was generated synthetically using the sequence shown in FIGS. 8A-B as a template. The nucleotide sequence encoding the β-farnesene synthase was amplified by PCR from its DNA synthesis construct using the primers shown in FIGS. 4U and 4V. To create a leader NcoI restriction enzyme site in the PCR product comprising the β-farnesene synthase coding sequence, the codon encoding the second amino acid in the original polypeptide sequence (TCG coding for serine) was replaced by a codon encoding aspartic acid (GAC) in the 5′ PCR primer (underlined in primer sequence shown above). The resulting PCR product was partially digested using NcoI, and digested to completion using SadI restriction enzymes, the reaction mixture was resolved by gel electrophoresis, the approximately 1.7 kb DNA fragment comprising the β-farnesene synthase coding sequence was extracted, and the DNA fragment was inserted into the NcoI Sad restriction enzyme site of the pTrc99A vector, yielding plasmid pAM373 (see FIG. 6 for a plasmid map).

B650 host cell transformants were selected on LB media containing 100 μg/ml carbenicillin. B651 host cell transformants were selected on LB media containing 100 μg/ml carbenicillin and 50 μg/ml kanamycin. B652 host cell transformants were selected on LB media containing 100 μg/ml carbenicillin and 35 μg/ml chloramphenicol. B653 host cell transformants were selected on LB media containing 100 μg/ml carbenicillin, 50 μg/ml kanamycin, and 35 μg/ml chloramphenicol.

Single colonies were transferred from LB agar plates containing host cell transformants to culture tubes containing 5 mL of LB liquid medium and antibiotics as detailed above. The cultures were incubated by shaking at 30° C. on a rotary shaker at 250 rpm for 30 hours, at which point cell growth was arrested by chilling the cultures on ice. The cells were stored at −80° C. in cryo-vials in 1 mL frozen aliquots made up of 400 uL ice cold sterile 50% glycerol and 600 uL liquid culture.

### Example 5

This example demonstrates the production of β-farnesene in the Escherichia coli host strains of Example 4.

Seed cultures were grown overnight by adding the 1 mL stock aliquot to a 125 mL flask containing 25 mL M9-MOPS and antibiotics as detailed above. The seed cultures were used to inoculate 250 mL baffled production flasks containing 40 mL M9-MOPS minimal medium, 45 ug/mL thiamine, micronutrients, 1.00E-5 mol/L FeSO₄, 0.1 M MOPS, 0.5% yeast extract, 20 g/L of D-glucose, and antibiotics at an initial OD₆₀₀ of approximately 0.05. Production cultures were incubated by shaking at 30° C. in a humidified incubating shaker at 250 RPM until they reached an OD₆₀₀ of 0.2 to 0.3, at which point the production of amorphadiene in the host cells was induced with 1 mM IPTG (40 uL of 1M IPTG added to the culture medium). Cultures were overlain with 8 mL dodecane to capture the β-farnesene. Samples were taken at various time points, and the β-farnesene concentration in the samples, as well as the OD₆₀₀ of the cultures, were measured at each time point. Dry cell weight (DCW) was calculated according to the following verified formula: DCW=OD₆₀₀×0.4.

Farnesene concentration was measured by transferring 100 uL samples of the upper dodecane layer of each flask to a clean tube, centrifuging the samples to separate out any remaining cells or media, layer-diluting 10 uL aliquots of each dodecane sample into 500 uL ethyl acetate spiked with beta- or trans-caryophyllene as an internal standard in a clean glass GC vial, vortexing the mixture for 30 seconds, and analyzing the ethyl acetate samples by gas chromatography-mass spectrometry (GC/MS). Analyses were performed on a Hewlett-Packard 6890 gas chromatograph/mass spectrometer in full spectrum scan mode (50-500 m/z). To expedite run times, the temperature program and column matrix were modified to achieve optimal peak resolution and the shortest overall runtime. A 1 uL sample was separated on the GC using a HP-5MS column (available from Agilent Technologies, Inc., Palo Alto, Calif.) and helium carrier gas. The oven cycle for each sample was 150° C. hold for 3 minutes, increasing temperature at 25° C./minute to a temperature of 200° C., increasing temperature at 60° C./minute to a temperature of 300° C., and a hold at 300° C. for 1 minute. The resolved samples were analyzed by a Hewlett-Packard model 5973 mass-selective detector. Previous mass spectra demonstrated that the β-farnesene synthase product was β-farnesene, and that β-farnesene had a retention time of 4.33 minutes using this GC protocol. Farnesene titers were calculated by comparing generated peak areas against a quantitative calibration curve of purified β-farnesene in caryophyllene-spiked ethyl acetate. For averaged results, experiments were performed using 3 independent clones of each host strain. The result that was one standard deviation away from the mean was discarded, and the average of the results obtained for the 2 remaining clones was graphed.

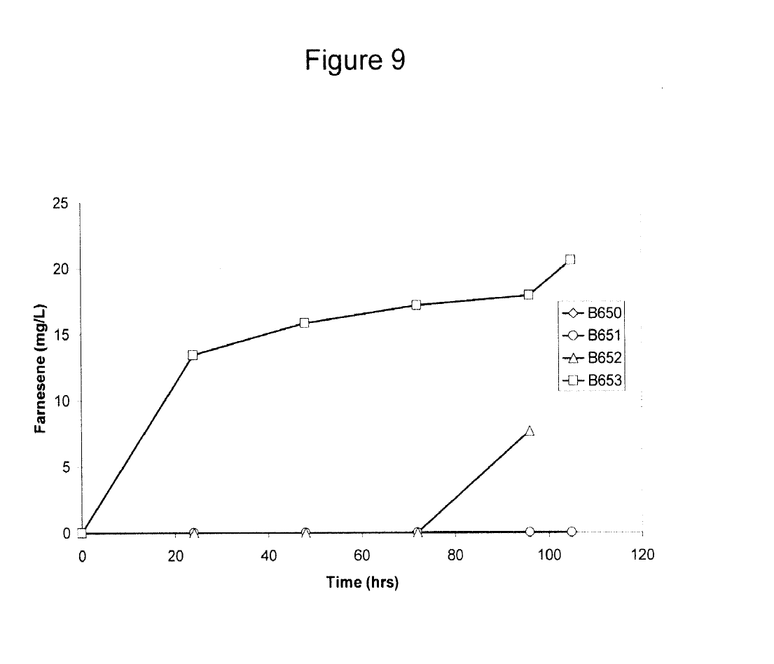

As shown in FIG. 9, Escherichia coli host strain B653, which comprises nucleotide sequences encoding enzymes of the full engineered DXP pathway, produced approximately 7 mg/g DCW β-farnesene.

What is claimed is:

1. A genetically modified host cell capable of converting pyruvate and glyceralderhyde 3-phosphate into dimethylallyldiphosphate (DMAPP), wherein the host cell comprises heterologous nucleic acid sequences that encode:
a. an enzyme that converts pyruvate and glyceraldehyde 3-phosphate to 1-deoxy-D-xylulose-5-phosphate (DXP);
b. an enzyme that converts 1-hydroxy-2-methyl-2-(E)-butenyl-4-diphosphate (HMBPP) to IPP, and,
c. an enzyme that converts IPP to DMAPP.

2. The host cell of claim 1 wherein the host cell additionally comprises one or more additional heterologous nucleic acid sequences that encode one or more enzymes selected from the group consisting of:
a. an enzyme that converts DXP to 2C-methyl-D-erythritol-4-phosphate (MEP);
b. an enzyme that converts MEP to 4-diphosphocytidyl-2C-methyl-D-erythritol (CDP-ME);
c. an enzyme that converts CDP-ME to 4-diphosphocytidyl-2C-methyl-D-erythritol-2-phosphate (CDP-MEP);
d. an enzyme that converts CDP-MEP to 2C-methyl-D-erythritol 2,4-cyclodiphosphate (MEC); and
e. an enzyme that converts MEC to HMBPP.

3. The host cell of claim 1 wherein the host cell comprises a heterologous nucleic acid sequence that encodes an enzyme that converts IPP and DMAPP into geranyl pyrophosphate.

4. The host cell of claim 1 wherein the host cell comprises a heterologous nucleic acid sequence that encodes an enzyme that converts IPP and DMAPP into farnesyl pyrophosphate.

5. The host cell of claim 1 wherein the host cell comprises a heterologous nucleic acid sequence that encodes an enzyme that converts IPP and DMAPP into geranylgeranyl pyrophosphate.

6. The host cell of claim 1, wherein the host cell has an endogenous MEV pathway.

7. The host cell of claim 4 wherein the host cell is S. cerevisiae.

8. The host cell of claim 1, wherein the host cell has an endogenous DXP pathway.

9. The host cell of claim 6 wherein the host cell is E. coli.

10. A genetically modified host cell having an endogenous DXP pathway comprising heterologous nucleic acid sequences wherein the heterologous nucleic acid sequences encode:
a. an enzyme that converts pyruvate and glyceraldehyde 3-phosphate to 1-deoxy-D-xylulose-5-phosphate (DXP);
b. an enzyme that converts DXP to 2C-methyl-D-erythritol-4-phosphate (MEP);
c. an enzyme that converts MEP to 4-diphosphocytidyl-2C-methyl-D-erythritol (CDP-ME);
d. an enzyme that converts CDP-ME to 4-diphosphocytidyl-2C-methyl-D-erythritol-2-phosphate (CDP-MEP);
e. an enzyme that converts CDP-MEP to 2C-methyl-D-erythritol 2,4-cyclodiphosphate (MEC);
f. an enzyme that converts MEC to 1-hydroxy-2-methyl-2-(E)-butenyl-4-diphosphate (HMBPP);
g. an enzyme that converts HMBPP to IPP, and,
h. an enzyme that converts IPP to DMAPP.

11. The host cell of claim 10 wherein the heterologous nucleic acid sequences are under the control of a single regulatory element.

12. The host cell of claim 10 wherein the host cell comprises a heterologous nucleic acid sequence that encodes an enzyme that converts IPP and DMAPP into geranyl pyrophosphate.

13. The host cell of claim 10 wherein the host cell comprises a heterologous nucleic acid sequence that encodes an enzyme that converts IPP and DMAPP into farnesyl pyrophosphate.

14. The host cell of claim 10 wherein the host cell comprises a heterologous nucleic acid sequence that encodes an enzyme that converts IPP and DMAPP into geranylgeranyl pyrophosphate.

15. The host cell of claim 10 wherein the host cell is E. coli.

16. A method for making an isoprenoid or isoprenoid precursor, comprising: culturing in an aqueous medium a genetically modified host cell capable of converting pyruvate and glyceralderhyde 3-phosphate into dimethylallyldiphosphate (DMAPP), wherein the host cell comprises heterologous nucleic acid sequences that encode:
a. an enzyme that converts pyruvate and glyceraldehyde 3-phosphate to 1-deoxy-D-xylulose-5-phosphate (DXP);
b. an enzyme that converts 1-hydroxy-2-methyl-2-(E)-butenyl-4-diphosphate (HMBPP) to IPP, and,
c. an enzyme that converts IPP to DMAPP.

17. The method of claim 16 wherein the host cell comprises one or more additional heterologous nucleic acid sequences that encode one or more enzymes selected from the group consisting of:
a. an enzyme that converts DXP to 2C-methyl-D-erythritol-4-phosphate (MEP);
b. an enzyme that converts MEP to 4-diphosphocytidyl-2C-methyl-D-erythritol (CDP-ME);
c. an enzyme that converts CDP-MEP to 2C-methyl-D-erythritol 2,4-cyclodiphosphate (MEG); and
d. an enzyme that converts MEG to HMBPP.

18. A method of making an isoprenoid or an isoprenoid precursor, comprising culturing in an aqueous medium a genetically modified host cell having an endogenous DXP pathway, wherein the host cell comprises heterologous nucleic acid sequences that encode:
a. an enzyme that converts pyruvate and glyceraldehyde 3-phosphate to 1-deoxy-D-xylulose-5-phosphate (DXP);
b. an enzyme that converts DXP to 2C-methyl-D-erythritol-4-phosphate (MEP);
c. an enzyme that converts MEP to 4-diphosphocytidyl-2C-methyl-D-erythritol (CDP-ME);
d. an enzyme that converts CDP-ME to 4-diphosphocytidyl-2C-methyl-D-erythritol-2-phosphate (CDP-MEP);
e. an enzyme that converts CDP-MEP to 2C-methyl-D-erythritol 2,4-cyclodiphosphate (MEC);
f. an enzyme that converts MEC to 1-hydroxy-2-methyl-2-(E)-butenyl-4-diphosphate (HMBPP);
g. an enzyme that converts HMBPP to IPP, and,
h. an enzyme that converts IPP to DMAPP.

19. The method of claim 18 wherein the heterologous nucleic acid sequences are under the control of a single regulatory element.

20. The method of claim 18 wherein the host cell is E. coli.
